(19) United States
(12) Patent Application Publication
(10) Pub. No.: US 2006/0040346 A1
(43) Pub. Date: Feb. 23, 2006

(54) Bacillus host cell

(75) Inventors: Jens Tonne Andersen, Naerum (DK); Steen Troels Jorgensen, Allerod (DK); Michael Dolbjerg Rasmussen, Vallensbaek (DK); Peter Bjarke Olsen, Kobenhavn O (DK); Ib Groth Clausen, Hillerod (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/510,941

(22) PCT Filed: Mar. 25, 2003
(86) PCT No.: PCT/DK03/00199

§ 371 Date: Oct. 8, 2004

(30) Foreign Application Priority Data
Apr. 10, 2002 (DK) PA 2002 00532

(51) Int. Cl. C07H 21/04; C12P 21/06; C12N 9/24; C12N 9/32; C12N 15/74; C12N 1/21

(52) U.S. Cl. 435/69.1; 435/200; 435/204; 435/252.31; 435/471; 536/23.2

(57) ABSTRACT

A Bacillus licheniformis mutant host cell derived from a parent B. licheniformis host cell, which mutant host cell is mutated in one or more gene(s) encoding one or more polypeptide(s) involved in antibiotic synthesis which is at least 80% identical to one or more of the polypeptides shown in SEQ ID NO's: 2 to 22, wherein the mutant host cell expresses at least 5% less of the one or more polypeptide(s) involved in antibiotic synthesis than the parent host cell, when they are cultivated under comparable conditions.

### TECHNICAL FIELD

Bacillus sp. are attractive hosts for the production of heterologous proteins due their ability to secrete proteins directly into the culture medium. They have a high capacity for protein secretion, are genetically highly amenable, nonpathogenic and free of endotoxins, and consequently a large variety of proteins from different organisms have been efficiently produced and secreted in Bacillus sp. ie. in Bacillus licheniformis.

In the highly competitive biotech industry, even slightly improved Bacillus host cells are in demand, which may provide more attractive production systems, or may even just be alternative production systems.

### BACKGROUND

Many industrial products of commercial interest can be produced biologically in Bacillus sp. host cells e.g. heterologous polypeptides, amino acids, carbohydrates etc. Some of these products are sold as process aids, intermediates; or even end-products in the food and feed industries as well as in the pharmaceutical industry. There are increasingly strict regulations that must be complied with when producing such products in microbial production hosts for sale in these industries, for instance traces of antibiotics in the products is seen as a problem. When producing in Bacillus licheniformis it is thus desirable to ensure that the host cell is not capable of producing antibiotic compounds native to the cell such as lichenysin, subtilisin, and surfactin.

### SUMMARY

A problem to be solved by the present invention is how to obtain a Bacillus licheniformis host cell incapable of producing native antibiotic compounds, or how to impair the production of these compounds in said cell. The present invention provides a solution to the problem by providing a Bacillus licheniformis host cell which has a reduced capacity to produce one or more polypeptide(s) involved in antibiotic synthesis.

Accordingly, in a first aspect the invention relates to a Bacillus licheniformis mutant host cell derived from a parent B. licheniformis host cell, which mutant host cell is mutated in one or more gene(s) encoding one or more polypeptide(s) involved in antibiotic synthesis which is at least 80% identical to one or more of the polypeptides shown in SEQ ID NO's: 2 to 10 (both included), or SEQ ID NO's: 2 to 22 (both included), preferably at least 85% identical, more preferably at least 90% identical, still more preferably at least 95% identical, and most preferably at least 97% identical to one or more of the polypeptides shown in SEQ ID NO's: 2 to 10 (both included), or SEQ ID NO's: 2 to 22 (both included), wherein the mutant host cell expresses at least 5% less of the one or more polypeptide(s) involved in antibiotic synthesis than the parent host cell, when they are cultivated under comparable conditions.

Preferably the mutant host cell expresses at least 10% less, more preferably at least 20% less, still more preferably at least 30% less, even more preferably at least 40% less, yet more preferably at least 50% less, or at least 60% less, or at least 70% less, or at least 80%, or most preferably at least 90% less of the one or more polypeptide(s) involved in antibiotic synthesis than the parent host cell, when they are cultivated under comparable conditions. Most preferably the mutant host cell expresses absolutely nothing of the one or more polypeptide(s) involved in antibiotic synthesis.

Comparable conditions of cultivation must be used in order to compare the expression level of the one or more polypeptide(s) involved in antibiotic synthesis in a mutant host cell of the invention with that in a parent host cell. They are cultivated separately under identical conditions in identical setups, of course allowing for the usual standard deviations of the operating parameters normally associated with growth experiments, such as temperature control etc. The quantification of the expression level of the one or more polypeptide(s) is done by standard text-book assay techniques as known in the art e.g. mRNA quantification or immuno-based assays.

In a second aspect the invention relates to a process for producing at least one product of interest in a Bacillus licheniformis mutant host cell, comprising cultivating a B. licheniformis mutant host cell as defined in the previous aspect in a suitable medium, whereby the said product is produced.

Finally, an aspect of the invention relates to a use of a Bacillus licheniformis mutant host cell as defined in the first aspect for producing at least one product of interest comprising cultivating the mutant host cell in a suitable medium whereby the said product is produced.

### DEFINITIONS

Nucleic acid construct: When used herein, the term “nucleic acid construct” means a nucleic acid molecule, either single- or double-stranded, which is isolated from a naturally occurring gene or which has been modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

Control sequence: The term “control sequences” is defined herein to include all components, which are necessary or advantageous for the expression of a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Operably linked: The term “operably linked” is defined herein as a configuration in which a control sequence is appropriately placed at a position relative to the coding sequence of the DNA sequence such that the control sequence directs the expression of a polypeptide.

Coding sequence: When used herein the term “coding sequence” is intended to cover a nucleotide sequence, which directly specifies the amino acid sequence of its protein product. The boundaries of the coding sequence are generally determined by an open reading frame, which usually begins with the ATG start codon. The coding sequence typically include DNA, cDNA, and recombinant nucleotide sequences.

Expression: In the present context, the term “expression” includes any step involved in the production of the polypeptide including, but not limited to, transcription, post-transcriptional modification, translation, post-translational modification, and secretion.

Expression vector: In the present context, the term uexpression vector” covers a DNA molecule, linear or circular, that comprises a segment encoding a polypeptide of the invention, and which is operably linked to additional segments that provide for its transcription.

### DETAILED DISCLOSURE

A Bacillus licheniformis mutant host cell derived from a parent B. licheniformis host cell, which mutant host cell is mutated in one or more gene(s) encoding one or more polypeptide(s) involved in antibiotic synthesis which is at least 80% identical to one or more of the polypeptides shown in SEQ ID NO's: 2 to 10 (both included), or SEQ ID NO's:. 2 to 22 (both included), wherein the mutant host cell expresses at least 5% less of the one or more polypeptide(s) involved in antibiotic synthesis than the parent host cell, when they are cultivated under comparable conditions.

The term “parent host cell” in the context of the present invention means a cell which is genetically identical, or isogenic, to the progeny mutant or mutant cell of the present invention, except for the mutated one or more gene(s) encoding one or more polypeptide(s) involved in antibiotic synthesis in said mutant.

The degree of identity, or %-identity of polypeptide sequences can suitably be investigated by aligning the sequences using a computer program known in the art, such as “GAP” provided in the GCG program package (Program Manual for the Wisconsin Package, Version 8, August 1994, Genetics Computer Group, 575 Science Drive, Madison, Wis., USA 53711)(Needleman, S. B. and Wunsch, C. D., (1970), Journal of Molecular Biology, 48, 443-453). Using GAP with the following settings for DNA sequence comparison: GAP creation penalty of 5.0 and GAP extension penalty of 0.3″.

An object of the present invention is to provide a culture medium free of antibiotics so as to be able to reduce the product purification to a minimum, and to comply with regulatory requirements. This may be done according to the invention by reducing or even completely abolishing the expression of one or more gene(s) encoding a native polypeptide(s) involved in antibiotic synthesis via mutagenisation of that (those) gene(s). One of the very well-known method of ensuring that a gene is not expressed into an active polypeptide within a cell is simply to delete or partially delete the encoding gene. Many techniques have been described in the art on how to specifically delete or partially delete one or more gene(s) in the genome of a cell, and certainly from the genome of a Bacillus licheniformis cell (see e.g. Novozymes A/S WO 01/90393, Novozymes A/S WO 02/00907). Accordingly, a preferred embodiment of the present invention relates to a host cell of the first aspect, which is mutated by a partial or complete deletion of the one or more gene(s) encoding the one or more polypeptide(s) involved in antibiotic synthesis.

A preferred embodiment of the present invention relates to a host cell of the first aspect, which is mutated in two or more genes encoding two or more polypeptides involved in antibiotic synthesis.

The product of interest to be produced by the mutant host cell of the first aspect may be one or more polypeptide(s) encoded by one or more heterologous gene(s). Consequently, a preferred embodiment of the present invention relates to a host cell of the first aspect, which comprises one or more heterologous gene(s) encoding one or more heterologous polypeptide(s).

In the industrial production of polypeptides it is of interest to achieve a product yield as high as possible. One way to increase the yield is to increase the copy number of a gene encoding a polypeptide of interest. This can be done by placing the gene on a high copy number plasmid. However, plasmids are unstable and are often lost from the host cells if there is no selective pressure during the cultivation of the host cells. Another way to increase the copy number of the gene of interest is to integrate it into the host cell chromosome in multiple copies. Integration of two genes has been described in WO 91/09129 and WO 94/14968 (Novozymes A/S) the content of which is hereby incorporated by reference. A preferred embodiment of the present invention relates to a host cell of the first aspect, wherein the heterologous gene(s) is present in at least two copies, preferably at least 4 copies, and most preferably at least 6 copies. In another embodiment the heterologous gene(s) is present in at least ten copies. If carried on a plasmid the gene(s) may be present in several hundred copies per cell, so in a still further embodiment of the present invention the heterologous gene(s) is present in at least 100 copies.

Integration of two genes closely spaced in anti-parallel tandem to achieve better stability has been described in WO 99/41358 (Novozymes A/S) the content of which is hereby incorporated by reference, as well as the stable chromosomal multi-copy integration of genes described in WO 02/00907 (Novozymes A/S) the content of which is incorporated herein by reference. A preferred embodiment of the present invention relates to a host cell of the first aspect, wherein the heterologous gene(s) are stably integrated into the genome of the cell.

Selection of chromosomal integrant has for convenience resulted in the use of selectable markers such as antibiotic resistance markers. However it is desirable if possible to avoid the use of antibiotic marker genes. WO 01/90393 discloses a method for the integration of a gene in the chromosome of a host cell without leaving antibiotic resistance markers behind in the strain, the content of which is hereby incorporated by reference A preferred embodiment of the present invention relates to a host cell of the first aspect wherein the heterologous gene(s) is integrated into the genome of the cell without leaving any antibiotic resistance marker gene(s) at the site of integration.

The present invention also relates to nucleic acid constructs comprising a nucleotide sequence encoding a product of interest, which may be operably linked to one or more control sequences that direct the expression of the coding sequence in a suitable host cell under conditions compatible with the control sequences.

A nucleotide sequence encoding a polypeptide of interest may be manipulated in a variety of ways to provide for expression of the polypeptide. Manipulation of the nucleotide sequence prior to its insertion into a vector may be desirable or necessary depending on the expression vector. The techniques for modifying nucleotide sequences utilizing recombinant DNA methods are well known in the art.

Other ways of increasing the product yield would be to increase promoter activity of the specific promoter regulating the expression of a specific gene of interest. Also a more general increase in the activity of several promoters at the same time could lead to an improved product yield. The control sequence may be an appropriate promoter sequence, a nucleotide sequence which is recognized by a host cell for expression of the nucleotide sequence. The promoter sequence contains transcriptional control sequences, which mediate the expression of the polypeptide. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

Other useful promoters are described in WO 93/10249, WO 98/07846, and WO 99/43835 (Novozymes A/S) the contents of which are incorporated fully herein by reference. A preferred embodiment of the present invention relates to a host cell of the first aspect, wherein the heterologous gene(s) are transcribed from a heterologous promoter or from an artificial promoter.

The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention.

The control sequence may also be a suitable leader sequence, a nontranslated region of an mRNA which is important for translation by the host cell. The leader sequence is operably linked to the 5′ terminus of the nucleotide sequence encoding the polypeptide. Any leader sequence that is functional in the host cell of choice may be used in the present invention.

The control sequence may also be a polyadenylation sequence, a sequence operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

The control sequence may also be a signal peptide coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GALL system may be used. In eukaryotic systems, these include the dihydrofolate reductase gene which is amplified in the presence of methotrexate, and the metallothionein genes which are amplified with heavy metals. In these cases, the nucleotide sequence encoding the polypeptide would be operably linked with the regulatory sequence.

The present invention also relates to recombinant expression vectors comprising the nucleic acid construct of the invention. The various nucleotide and control sequences described above may be joined together to produce a recombinant expression vector which may include one or more convenient restriction sites to allow for insertion or substitution of the nucleotide sequence encoding the polypeptide at such sites. Alternatively, the nucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about the expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome.

The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

Examples of bacterial selectable markers are the dal genes from Bacillus subtilis or Bacillus licheniformis, or markers which confer antibiotic resistance such as ampicillin, kanamycin, chloramphenicol or tetracycline resistance.

The vectors of the present invention preferably contain an element(s) that permits stable integration of the vector into the host cell's genome or autonomous replication of the vector in the cell independent of the genome.

For integration into the host cell genome, the vector may rely on the nucleotide sequence encoding the polypeptide or any other element of the vector for stable integration of the vector into the genome by homologous or nonhomologous recombination. Alternatively, the vector may contain additional nucleotide sequences for directing integration by homologous recombination into the genome of the host cell. The additional nucleotide sequences enable the vector to be integrated into the host cell genome at a precise location(s) in the chromosome(s). To increase the likelihood of integration at a precise location, the integrational elements should preferably contain a sufficient number of nucleotides, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell.

Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus. The origin of replication may be one having a mutation which makes its functioning temperature-sensitive in the host cell (see, e.g., Ehrlich, 1978, Proceedings of the National Academy of Sciences USA 75: 1433).

More than one copy of a nucleotide sequence of the present invention may be inserted into the host cell to increase production of the gene product. An increase in the copy number of the nucleotide sequence can be obtained by integrating at least one additional copy of the sequence into the host cell genome or by including an amplifiable selectable marker gene with the nucleotide sequence where cells containing amplified copies of the selectable marker gene, and thereby additional copies of the nucleotide sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

A preferred embodiment of the present invention relates to a host cell of the first aspect, wherein the heterologous gene(s) are comprised in an operon, preferably a polycistronic operon. The term “operon” in the context of the present invention means a polynucleotide comprising several genes that are clustered and perhaps even transcribed together into a polycistronic mRNA, e.g. genes coding for the enzymes of a metabolic pathway. The transcription of an operon may be initiated at a promoter region and controlled by a neighboring regulatory gene, which encodes a regulatory protein, which in turn binds to the operator sequence in the operon to respectively inhibit or enhance the transcription. The gene or the operon can be carried on a suitable plasmid that can be stably maintained, e.g. capable of stable autonomous replication in the host cell (the choice of plasmid will typically depend on the compatibility of the plasmid with the host cell into which the plasmid is to be introduced) or it can be carried on the chromosome of the host. The said gene may be endogenous to the host cell in which case the product of interest is a protein naturally produced by the host cell and in most cases the gene will be in it normal position on the chromosome. If the gene encoding the product of interest is an exogenous gene, the gene could either be carried on a suitable plasmid or it could be integrated on the host chromosome. In one embodiment of the invention the eubacterium is a recombinant eubacterium. Also the product of interest may in another embodiment be a recombinant protein.

The product of interest is any gene product or product of a metabolic pathway which is industrially useful and which can be produced in a bacterial cell such as a B. licheniformis.

In one preferred embodiment, the heterologous polypeptide(s) is an antimicrobial peptide, or a fusion peptide comprising a peptide part which in its native form has antimicrobial activity.

In another preferred embodiment, the heterologous polypeptide(s) has biosynthetic activity and produces a compound or an intermediate of interest.

Yet another embodiment relates to a host cell of the first aspect, wherein the compound or intermediate of interest comprises vitamins, amino acids, antibiotics, carbohydrates, or surfactants, and preferably the carbohydrates comprise hyaluronic acid.

In one embodiment the heterologous polypeptide(s) is an enzyme, particularly the enzyme is an enzyme of a class selected from the group of enzyme classes consisting of oxidoreductases (EC 1), transferases (EC 2), hydrolases (EC 3), lyases (EC 4), isomerases (EC 5), and ligases (EC 6). Preferably the enzyme is an enzyme with an activity selected from the group consisting of aminopeptidase, amylase, amyloglucosidase, mannanase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, or xylanase. Preferably the enzyme is an amylase or a mannanase.

A second aspect of the invention relates to a process for producing at least one product of interest in a Bacillus licheniformis mutant host cell, comprising cultivating a B. licheniformis mutant host cell as defined in the first aspect of the invention in a suitable medium, whereby the said product is produced. One embodiment relates to a process of the second aspect, further comprising isolating or purifying the product of interest. Suitable media for the cultivation is described below as well as methods for the purification or isolation of the produced product which is an optional additional step to the process of the present invention.

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or Industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The medium used to culture the cells may be any conventional medium suitable for growing the host cells, such as minimal or complex media containing appropriate supplements. Suit-able media are available from commercial suppliers or may be prepared according to published recipes (e.g. in catalogues of the American Type Culture Collection). The media are prepared using procedures known in the art (see, e.g., references for bacteria and yeast; Bennett, J. W. and LaSure, L., editors, More Gene Manipulations in Fungi, Academic Press, CA, 1991).

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered by methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

A third aspect of the present invention relates to the use of a Bacillus licheniformis mutant host cell as defined in the first aspect for producing at least one product of interest comprising cultivating the mutant host cell in a suitable medium whereby the said product is produced, and optionally isolating or purifying the produced product.

1-21. (canceled)

22. A Bacillus licheniformis mutant host cell derived from a parent B. licheniformis host cell, which mutant host cell is mutated in one or more gene(s) encoding one or more polypeptide(s) involved in antibiotic synthesis which is at least 80% identical to one or more of the polypeptides shown in SEQ ID NOs: 2 to 22, wherein the mutant host cell expresses at least 5% less of the one or more polypeptide(s) involved in antibiotic synthesis than the parent host cell, when they are cultivated under comparable conditions.

23. The host cell of claim 22, which is mutated by a partial or complete deletion of the one or more gene(s) encoding the one or more polypeptide(s) involved in antibiotic synthesis.

24. The host cell of claim 22, in which two or more genes encoding two or more polypeptides involved in antibiotic synthesis are mutated.

25. The host cell of claim 22, which comprises one or more heterologous gene(s) encoding one or more heterologous polypeptide(s).

26. The host cell of claim 25, wherein the heterologous gene(s) are present in at least two copies.

27. The host cell of claim 25, wherein the heterologous gene(s) are stably integrated into the genome of the cell.

28. The host cell of claim 25, wherein the heterologous gene(s) are integrated into the genome of the cell without leaving any antibiotic resistance marker genes at the site of integration.

29. The host cell of claim 25, wherein the heterologous gene(s) are transcribed from a heterologous promoter or from an artificial promoter.

30. The host cell of claim 25, wherein the heterologous gene(s) are comprised in an operon.

31. The host cell of claim 25, wherein the heterologous polypeptide(s) are antimicrobial peptides and/or a fusion peptide comprising a peptide which in its native form has antimicrobial activity.

32. The host cell of claim 25, wherein the heterologous polypeptide(s) have biosynthetic activity and produce a compound or an intermediate of interest.

33. The host cell of claim 32, wherein the compound or intermediate of interest comprises vitamins, amino acids, antibiotics, carbohydrates, or surfactants.

34. The host cell of claim 33, wherein the carbohydrates comprise hyaluronic acid.

35. The host cell of claim 25, wherein the heterologous polypeptide(s) are enzymes.

36. The host cell of claim 35, wherein the enzymes are enzymes of a class selected from the group of enzyme classes consisting of oxidoreductases (EC 1), transferases (EC 2), hydrolases (EC 3), lyases (EC 4), isomerases (EC 5), and ligases (EC 6).

37. The host cell of claim 36, wherein the enzymes are enzymes with an activity selected from the group of enzyme activities consisting of aminopeptidase, amylase, amyloglucosidase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannanase, mannosidase, oxidase, pectinase, peroxidase, phytase, phenoloxidase, polyphenoloxidase, protease, ribonuclease, transferase, transglutaminase, and xylanase.

38. The host cell of claim 37, wherein the enzymes are amylases or mannanases.

39. A process for producing a product of interest, comprising cultivating the B. licheniformis mutant host cell of claim 22 in a suitable medium to produce the product of interest.

40. The process of claim 39, further comprising isolating or purifying the product of interest.


SEQUENCE LISTING (22 sequences)

SEQ ID NO: 1 - DNA, 1602 bp, Bacillus licheniformis
  CDS (501)..(1421)
aacagccagaaaggagacgaagaacgtctctatttaacagagcttgatatgttctgcgtccatttcgggtcaaggatatattcactattatgaaaccaatttttccgatttccgtataaacaaagcgtggaacctgttaaaggacaagaaaaatcgacagggatataaaaatcggcaagctttccaaagggaaccggggcaggctgaagctagcgctcacattggcgagcgatgcagcggtcgtgctgtttgatgaaccatccggtctccgatggtgagggagtcgacagtcaaaagccttcttacatatttggagcttgacaagcagacaatcgccatcgccacctatgaaattgaacaaatattggatgtcgtcttaaatggccgaattgaagcaaaaaaggatgtcgaacagctgcgcgaggaatccggaatgtcgcttttggaatggctgaaacggtttattaaagaaaaaaattgaaaaagcgggaggagaaacattggaaacacttttggaattaaaaaatgtatcaaaaacgatcagggggaaaaagatcatcgagggcttgagttttgacgtgcgggcaggcgagatattcggcttcctggggccgaacggcgccggaaaaacgacgacgatccggatgattgtcggacatatgagcattaccgccggtgaaatcgccgtgtgcggcgtcagcgtaaaagaaaactttgaaaaggctgcacggcatatcggagcgatcgttgaaaacccggagctttataaatttttaacgggctaccaaaaccttcagcaatacgcgcgcatgacgaaaggcgtgacgaaaaagaaaattgacgaaatcgtcgagctcgtcggattgaaaaacaggatcaacgacaaggtcaaaacgtattcgttaggaatgagacaaaggcttggccttgcccaaagccttttgcacgatccaaagctgttgattctcgatgagccgacgaacgggcttgatccggcaggtatccgggaaatccgtgactatttaagaaagctgacgagagaaaagggaatggcggtcatcgtttcaagccacctgctttcagaaatggagctgatgtgcgacaggatcgccattattcaaaacggaaagctccgggatattcagcatgtgcacggacccgctcgggatgagaagaagcgctattatattcaagcggacgacacccaggctctcacacgggaagcggctgctttcagaaaggtgaaggttgacgaagcggaaggcgggatagagctcagcattcaaaaggatgaagtgcctgatttgattaaacacttgacagacagcggtgttcgcttatatgaagtgaaggctgtgaacaaatcgctggaagaccgattcctggaaatcaccgcagataaggaggaagctcagcatgtttaatctcatcgtaaatgaatggatcaaaatttttaatcgaaaaggcacatacgtcatgatcggaattttgctgttagctgtcatcgggctgggcgttctcacaaagacgatcggagagacagaccaaaacacggactggaaaaaggaattggcgcaggaaataaggacaagggggcttagt

SEQ ID NO: 2 - protein, 307 aa, Bacillus licheniformis
LETLLELKNVSKTIRGKKIIEGLSFDVRAGEIFGFLGPNGAGKTTTIRMIVGHMSITAGEIAVCGVSVKENFEKAARHIGAIVENPELYKFLTGYQNLQQYARMTKGVTKKKIDEIVELVGLKNRINDKVKTYSLGMRQRLGLAQSLLHDPKLLILDEPTNGLDPAGIREIRDYLRKLTREKGMAVIVSSHLLSEMELMCDRIAIIQNGKLRDIQHVHGPARDEKKRYYIQADDTQALTREAAAFRKVKVDEAEGGIELSIQKDEVPDLIKHLTDSGVRLYEVKAVNKSLEDRFLEITADKEEAQHV

SEQ ID NO: 3 - DNA, 1938 bp, Bacillus licheniformis
  CDS (501)..(1457)
cgcatcggacttttgaacgtcggaaccgaagataaaaaaggaaatgagcttgccaagcagacctttcaaaaattgaaggaaaccgatttgaatttcatcggcaatgtggaagcccgcgatatgctggacggagtcgctgatgtcatcgtcacagacggctttaccggtaacgttgccttgaaaacggtcgagggcgcggcgctgtccatttttaaaatgctgagaacgacgctgacttcgagcttcacggcgaagctcgccgcttctgcactgaagccgaagctgaaagaaatgaaaacgaaaatggattactctgaatacggcggagccggattgttcggcttaaaggcgcccgtcatcaaagcgcacggatcatctgacggacgcgccgtttatcacgcgatccgccaggccagagagatggtcagccaaaatgtcgcggcatttatcgaagaaaaaattcaacaaaaagcagatgaatagtctggaggttttaacacatgggcaagattgcttttctattcccgggccaaggttcgcagcatatcggcatgggacacgaattgtatgaaaaagaaccgaatgcgaagaagatttttgaagaagcggatcaaacgcttgaaacaaaactgagcaccctcatgtttgaaggggatgcaaaggaactgacgcttacatacaacgcgcagccaagccttttaacggcgagcatcgcagcgcttgaaaaactgaaggaatacggcattaaagccgactatgcggcaggtcacagcctcggcgaatacagcgcattggtcgctgccggcgccttgtcgtttaaagatgcggtttatgccgtcagaaagcgcggcgaattcatgaatgaagccgtgccggcgggagaaggcgcgatggcggccattctcggcatggacagccaggcgctgaaagaagtgacggacaaaatttccgaagaaggaaaccttgttcagctcgccaatttgaactgccctgggcaaatcgtcatctcgggaacagctaaaggcgtggagctcgcttcagagcttgcgaaagaaaagggcgcaaaacgcgcgattcctctcgaagtcagcgggccgttccattctgagctgatgaagccggcagctgataagcttcgtgaagttcttgatgcgtgcacgatcaacgacgcagccattccggtcgtctccaacgtaacggccgactttgtaacggataaagacgacattaagaataaactgattgaacagctgtattcccctgtacgctttgaagaaacaatcagccgcctgattgacgaaggcgtcacgaccttcattgaaatcggtcccggaaaggttttgtcagggcttgtgaagaaagtgaaccgcagagtcaaaacgattgctgtatcagacccgaacacaattgaacttgccgttcaaacgttgaaggaggaaaacgaaaatgctggaaaataaaacagccgttgtgacaggagcctcaagaggaatcggccgcgcgatcgccctggacctggcgaaaaacggagcaaatgtcgtcgtcaactacgcgggaaatgaagcgaaagcgaacgaagtcgtagacgaaatcaaagcgctcggccgcgatgcgtttgcttttaaagcggacgtttccaatgcggatgaggttcaggcgatgatgaaggaagcggtcggacgcttcggcacgcttgacatccttgtcaacaatgcgggcattactaaagacaatctgttcatgagaatgaaagaagatgaatgggacgacgtcattaacataaacttaaaaggtgtgttcaattgttcaaaagctgtgacaagacagatgatgaaacaaagaagcggccggatcatcaatatcacctcggttgtaggcgtcgtcggtaacgccgggcaggccaactatgtcgcggctaaatcaggcgtattccagtag

SEQ ID NO: 4 - protein, 319 aa, Bacillus licheniformis
MGKIAFLFPGQGSQHIGMGHELYEKEPNAKKIFEEADQTLETKLSTLMFEGDAKELTLTYNAQPSLLTASIAALEKLKEYGIKADYAAGHSLGEYSALVAAGALSFKDAVYAVRKRGEFMNEAVPAGEGAMAAILGMDSQALKEVTDKISEEGNLVQLANLNCPGQIVISGTAKGVELASELAKEKGAKRAIPLEVSGPFHSELMKPAADKLREVLDACTINDAAIPVVSNVTADFVTDKDDIKNKLIEQLYSPVRFEETISRLIDEGVTTFIEIGPGKVLSGLVKKVNRRVKTIAVSDPNTIELAVQTLKEENENAGK

SEQ ID NO: 5 - DNA, 2119 bp, Bacillus licheniformis
  CDS (381)..(1616)
tagtggatcatacaatcatgaatggcagagaagtgttcaagtttgccgtccgccaaatgggtgaatccagtgtaaatgttattgaaaaagcgggattatccaaagaggatgtcgactttttggtgccgcaccaagccaacatccggatcatggaagcggcgcgcgaacgtttggagcttccggtcgaaaaaatgtcgaaaacggttcataaatacggaaacacatcagctgcttctattccgatttctcttgtggaagagcttgaagctggtaaaataaaagacggtgacgtcattgtgatggtcggcttcggcggaggtttgacatggggagccatcgccatgcgctggggccgctaaaagaaaggtgaggtgcaacttatgaatagtaaaagagtagtcattaccggattaggagctttatcacctcttggtaatgacgtcaaaacaagctggaacaacgcgatcaacggggtttccggagtcggcccgataacaagggtgaacgcggacgagtatcctgctaaagtagccgcagaactgaaagattttaagatagaagattatatggataaaaaagaagcgagaaaaatggaccgcttcacacaatatgccgttgtgtcggcgaaaatggccgtggaagacgcgggcctcgacattaacgaaagcaacgcagaccggatcggcgtctgggtcggttccggaatcggcggccttgaaacgcttgaacagcagttcgaaacatttttaacaaaaggaccgcggcgcgtcagcccgttctttgtaccgatgatgattcctgatatggcgaccggccagatttcgatctctctcggtgcaaaaggggtcaactcttgtacggtgacggcttgcgcgacgggtacaaactcaatcggcgacgctttcaaggtcattcagcgcggcgatgccgatgtcatgatctccggaggaacggaagccccgcttacaagaatgtcatttgccggcttttcagccaataaagcgctatccactaatccggatccgaaaacggcaagccgtccgtttgacaaaaaccgcgacggcttcgtcatgggagaaggagctggaatcgtcgttcttgaggagcttgagcacgccttaaaccgcggagcgacaatctacgcggaagtggtcggctacggatcaacgggagacgcctatcatattacggcacctgcgccaaatggagaaggcggcgtcagagccatgaaagaagcgatcaaagatgcgggtctgtcccctgtggacattgattatatcaatgctcatggaacaagcacgccgtacaatgataaatttgaaacgatcgccattaaagaagtgttcggagagcatgcctacaagcttgccgtcagctcgacaaaatcgatgacagggcacttgctcggggctgccggaggcatcgaagcgattttctccgtccttgccattaaggaaggcatcattcctccgacaatcaatatcgaaacgcctgatgaagattgcgatctggactatgtaccggatcaagcccgccgccaggacgtgaatgttgtgctcagcaactcgcttggtttcggcggacacaacgcaacgctgatcttcaaaaaatatgaataacccgcaatataaacggctgccattttaaatggtggccgttttttttagcgtgcataccatacagatgaaaaaggagtgtgatctaaaatgagtatagcaagcacgtatgaatggcttgaacaggcttcatccgtgcgggaattgccaagctttctcctgccgtattttaaaggcgcttcaaaagcggatgcggcggcgattgtcagccacctgcagatccacggcttgttccgttcgtggacggacggcgaacgtacgatgaagcagcttaaggaaaacggcgtatttcagcatgtcagacgggaagaacagctgctctgcaaaagatggaacgggccggatgttccgattttcatcctgccggtcgatgaaagaaaccgcaaaatccgcgtcgaattcggctcgaaatcaggcctcgcgttcagcgacaaattatttttgtttttatcggccgatcttccccggtcttcaatttctgcgatcatgacacacgaatataa

SEQ ID NO: 6 - protein, 412 aa, Bacillus licheniformis
MNSKRVVITGLGALSPLGNDVKTSWNNAINGVSGVGPITRVNADEYPAKVAAELKDFKIEDYMDKKEARKMDRFTQYAVVSAKMAVEDAGLDINESNADRIGVWVGSGIGGLETLEQQFETFLTKGPRRVSPFFVPMMIPDMATGQISISLGAKGVNSCTVTACATGTNSIGDAFKVIQRGDADVMISGGTEAPLTRMSFAGFSANKALSTNPDPKTASRPFDKNRDGFVMGEGAGIVVLEELEHALNRGATIYAEVVGYGSTGDAYHITAPAPNGEGGVRAMKEAIKDAGLSPVDIDYINAHGTSTPYNDKFETIAIKEVFGEHAYKLAVSSTKSMTGHLLGAAGGIEAIFSVLAIKEGIIPPTINIETPDEDCDLDYVPDQARRQDVNVVLSNSLGFGGHNATLIFKKYE

SEQ ID NO: 7 - DNA, 8158 bp, Bacillus licheniformis
  CDS (501)..(7655)
atcaccggaatctatgcccatatcggctgcctgatgacggcatgtgatgcttttatgcaagatattgaaacatttttcgtcggagatgcggtggcggatttctcattaaagcatcatgaaatggccatcacatacgcagctgaccgttgcagtgtcacaattactgcggaacagcttctgagccgactgaaagatggcgaaagccatgaacacgcaaaagaaagctctcatgcactgacaaaacagattattcgcgaacaggtggcatcgcttcttcttgaatcgccggaaaagataagcgacagcgaaaatttgatctaccggggaattgattccgtgagaatcatgagtctcgcagagcgctggcgccgagcggggcgggaggtgtcgtttgtagagcttgcggaggacccttcgattgaaaactggtggagactgttgtcctcctctaaagaagcacctttgccaaatgcagactatcaatgaaggaggtcacctaaatgcctgaatgtcaacataaccgaaagccattatcaggagcgcaagccgggatttggtttgctcagcagcttgatccggaaaatccgatctacaatacagctgaatacgttgaaattaaaggcccgcttgatcaggagcttttcgaaaaagcgctgcgccatgtcattaaagaagctgaatcatttcatgccagatttggagaagatcaagacggaccatggcaagagatcgttccgtcaacagattttccgctacattacattgatgtcagctcagaaaccgatccggaacaggcggctaaaagctggatgatggatgaccttgcccgtccggttgatctgactcgcggcccgctttttacagaagcgctttttaaagcggcgcaagatcattacttctggtatcagcgaactcaccatatcgcaacagacgggttcagctttacattgatcgccgagcggctgtcaaaaatatataccgcattgatgcagaacaaatcgatcgaccagagcggagcctttggctccctcgatttgattctcgcggaggaaacggcttaccgtgcatcagaacagtatcaggaagatcggcgattttggcttgggcgtttcagcgatgaaccggaggtggtcagccttgccgaaagggcgccgcgtacctcttcaagttttcttcgcaggtctgaacacctgccaagtgaggatgctgatcgtcttctgtctgccgcgagcagaatgggggcaacttggcacgaaaccgtgatggcagcagcagcaatatatgttcaccgtctgacaggggcaaatgacgtcgttctcgggatgccgatgatgtgccgcctcgggtcagccgctcttcatattccgggaatggtcatgaatctcctcccgcttcgaattggcatgcagccgcaaatgagcataggagagctagtcaagcaaatctcgggtgagatgatgaagctgcggcgccatcagcattaccgccacgaagaattgcgccgggatctcaagctgcttggcgaaaaccagcggctgttcggtccgcagcttaatttgatgccttttgagaaccgcttgaattttgccggctgtcaaggcatcgtgcataatcttgccacgggacctgtggacgatttgtcaatcaacatttacggccgtccggacggtggcgggctaaaagtaaacatggatgcaaaccctgctgtttatcatgcggatgaacttgaagatcacggcaatcgttttcttactcttctgaaaacgattgccgtttgcgaacaaactcagccggtggggacgttggacattcttctccctgaagaacgcgctcaaatattgattgaatggaatcaaacagaacacgggcttcccaaagagagtcttccgcagcgatttgaaaggatggcaaaggagtgtcctgaatctccggcagtcgtttgcaatgataaggttctcacttattccgaattaaatcaaaaggctaatcaactggctcacttgctgatcgatcagggggcgaaacccgagacatttattgccttggcgttgccgcggtcagcggaaatggttgtcagcatgctcgccgttctcaaagctggagcggcttatttacctatagatcccgattatccggctgacaggatcgagtatatgctcaacgatgcacagccgctgcttgttatgaccagcaaggaagcgcaagacacgatcggttcgcaaatgccgaagttaatcctcgatgaacaaactgttatggagaaaatgtccggctgttctgaagaaaatcccggcgaacagcattccggcaaccagccggcatatatgatttatacgtcgggatcaaccggcagacctaaaggcgtggtcgtacaagctgaaagcttattcaattttttgctgtcaatgcaggacatgttcgcgcttaatcaagatgaccggctgctggccgtcactacaattgcattcgacatctcagcactcgaaatttatttgccgctgatcagcggatcagcagtcgttcttgccgagaaggaaacggtacaagatccgtccgaattggccaaaatgattgaaacatacgaaattacaataatgcaggctacaccgaccctctggcatgcattggcctcgagcgccccggaaaagctcaaaggtcttcgtgcgcttgtcggcggcgaagctttgcaatccagcctggcccggcaattgcagcagctcgcgtgttctttaacgaacctttatggaccgacggaaacaacaatttggtctacagccgctgcgctggaaggcaactgtacggaacctccgggaatcggctgtgcgatttggaatacgcagctttatgtcctggacgccggattacagccagtgcctccgggaacggctggagaactttatgtggcaggaacaggggtagcgcgcggctacttgaaccgacatgctctcacggctgagcgctttattgcgaatccatacgggccgcctggatcgcggatgtaccggacaggcgacatcgtgcgctggcgcgaagacgggtcgcttgattatatcggccgtgcagaccatcaagtcaaaattcgggggttccgaattgaaatgggagaaatagaagcggtgcttgcaaatcatccggttgttaaacaagctgctgctatcgttcgtgaagaccagcccggtgacccgcgtttattcgcgtatgccgttcccgcttcgggagaaagccttgatcctgccgagcttcgccgcttcgttggtgagacactgcccgactatatgattccgtctgcatttgtcatacttgatgaactgccgcttacgccgaacggaaagcttgaccgcaaatccctgccggctccggcggtgagtatgcatacaggcggacgcgagccgaggactccgcaagaggaaattttgtgtgatttatttgctgaggtgctgggggtgccgcgagtcagtattgatgacagcttttttgacctcggcggacattcccttctggcaggcaggctcgtcggccgcattcgggaaatgctcggcgtcgaactcggaatcggccgtttattcgatgagcccacagccgccggacttgccaaacagcttgatcaggcgcagagcgcccgtccggcattgcgcaaaagagagcgccgcaaggagattccgctgtccttcgcacagcggcgcctatggtttttgcactgtttggaaggaccgagcccgacctataatattccggttgtcgttcatttaactggagatttggaccaaaaggcgctggcagctgctctgggtgatgtggcaacaagacatgagccgcttcgaacaattttcccggatcagcaagggacaacacaccagctgatattggaagaggatcaatcgcgtcccgaactcactgtatcccatgtcagcgaacatgagttggaaaaagtcctggctgaggccgtccggcaccgttaccatttagaaaaggagcctccgtttcgcgcccagctattcgtactcggaccggacaaatttgtgcttcttctgcttttgcaccatatgatcggcgacggctggtctttaatgccgctgacccgtgatttggaaaccgcctataatgcgcgtctgcaaggtgaggcacctgtttgggagccgctttctatacaatatgccgactatgccgtatggcaggaatatctgcttggcagtgagaacaatccggacagtctgattgccaaacagctcgaatattggtcgaaggcattggaacatctgcctgatcagctggagcttccgactgatcacccgcgtccgtcggaatctagctacagaagcggtacgattgatctcagcattgacgaacagctgcacggccgcctgttcgatttgtcccgcagcaccggagtcagcatgtttatgattttgcagtcggctcttgccgcactactgacacggctcggagcaggccatgatattccgctcggcagcccgatagcgggaagaaacgatgatgctttaggcgaaattgtcggattgtttgttaatacgcttgtgcttcgaacagatacatcgggtaatccgagttttcgggagcttctgaacagagtcagaaaagtaaaccttgcagcgtatgagcatcaggaccttccgttcgagcgtcttgttgaagtgcttaatccgcgcaggtcaagagcgagacatccgctgttccaaattatgcttgcctttcaaaatacgcctgaacctgagcttgacctttccggcctcaagtcagacattgaaattcgcagcgtcggagcagccaagtttgatttgacgattgagcttcgcgaacatcgaaaagcggacggaacacccgcaggtatcggcggatttcttgaatacagcacagatttatttgagcgaaatacggttcaaacattggcagaacggcttgtcaggctgctggattcagcagcagacgaccctgatcagccgatcgaaaaactcgatattctccttccggccgaacgggaaaacatgctggctgattggagcaaatcgtccaacagcataccttgttcatctttgcccgttttgtttgaaaagcaggcggcaaaggatccggaagctgttgcggttatatgtgaaaataatgcgctcacatacggcgagcttaataaacgcgctaaccggcttgctcatctcctgatcgccaaaggggtcggccccgaacaatttgctgccctggcgctgccaagatctttggatatggttgtcggattgcttgcagtgctgaaggctggcgcggcatatgtgcccctagatccggactacccggcggaaaggatagcgtttatgttgaatgatgcccatcctgtctgcatcgtaacgagttcagcggtagaatcaaatctttctgttcccggatctgttgaaaggattgtactcgatgatccttgtatacaagaggaactgaaaggatgcgctgctgcgaatccgtgtgatgccgaccgcaccgcgcctttgctgcctcttcacccggcgtatgtcatttacacatccggttcgaccggcaagccgaaaggagtcgttgttccgcatcaaaatgtcgtgcggctgttcggagcgactgaccaatggttccatttcggcgctgacgatgtttggacgatgtttcattcttacgcatttgacttttcggtatgggaaatttggggagcacttctcaatgggggccggcttatcgtggtgcctcatacaatcagccgctcgccggccgaatttctcaatcttctcgttcgcgagggagtgactgtgctcaatcagactccttcagcattttatcaattgatgcaggctgaccgggacaatgctgagacaggaaagctcctttccttgcgctttatcatttttggaggggaagcgctcgagctgaagaggcttgaagattggtatgaacgccatccggatcatttcccgcggctgattaatatgtatggaattacagaaacgacggtgcatgtcagctatatttctctcgatcagcagactgcggcacttcaagcgaacagcctgatcggccaggggattcccgatttaggtgtatatgtgcttgatgagtatcttgaaccggtgcccccgggcgtcaccggagagatgtatgtgtccgggggcggtctcgcacggggttacttgggcagacccgatttgacagcagaccgctttgtcgctgatccgtttggtccgccgggaacccggatgtaccgcaccggcgatcttgcccgcaggcgccaggacggctctctggattatatggggcgggccgaccagcagatcaagatccgcggcttcagaatcgaactcggcgagatagaagcggtgcttgtccgccatcatagagtgaatcaagccgctgtggtcgtaagggaaggccagcccggggacaagcgtctgatcgcctacgttgtcccggcgtctgaagaagaaaccgatccggctgagctgcgacggtttgccgcaggcactctgccggagtacatggtgccttccgcatttgtaaaaatttcagagctgccattgactccaaatggcaaacttgaccgaaaagcgctgcctgagcctgactttgccgcggctgttaaagggcggggcccaagaacaccgcaggaagaaatcctgtgcgatcttttttcagaaatcctcaatgcgccgagagtcggtattgatgacggattcttcgaacttggcgggcattctcttctcgccgtacagctgatgagccgcattcgtgaagcgctcggagtcgaactcggcatcggcgacctgcttgaagcgcctacagtcagcggacttgctgaacggcttgaatccggcggcaggcaaagtgcgctggatgtaatgcttccgctgagaaccggaggcagtcaagatcccctgttctgtgtgcacccggccggaggcctcagctggtgttatgccggtttaatgacggcactcggcaaggaatacccgatatacggccttcaagcccgcgggatcgcaaggcaggaagagctacctgatacgctcgacgacatggctgctgattatattcggcacattcgtacgattcagccaacaggcccttatcgtctgctcggctggtccctcggaggcaatgttgttcatgcgatcgcaactcagctgcaggaacaaggggaagacatatcgcttcttgtcatgcttgatgcttacccgaatcacttccttccaattaaagatgcgcctgatgagcaggaagcattgattgccctcttggcattgggaggatacgacccggacagccttgacggggcaccgctgaacctttccagtgcgatcgatattctgcgccgcgacggcagtgcgcttgccagcctggacgaggctgcgatcttgaacttaaaggagacgtatgtgaattccgtccggattttaagcgaatataagccccgcgtctttcatggcgatattctgtttttcaaatcaaccgtgataccggaatggtttgatccgatcgatccggagtcatggctaccttatttgaacggaaacattgatattcatgatatggattgccggcataaagatttgtgccagccggagccgctggccgaaatcggacgtcgggtttcggaaaagctggacgacttgaaaaaagatacggataagtagggagagatagcgatgacaaatccttttgaaaatgaacacggcacatatgtcgttttaatgaatcaggatggccagtattcactctggccggccttcattcatattccggcagggtgggaggtcgtctgcggggaagcgagccgcagcgcctgtatcgactatatcagctcgaattggaccgatctctcgccgaacagcatcaagctggttgcagaagtacacggcggtcagcaatgaatagaaggccgttcaatcaaaggacggttgtcagcattgtatatgtaacagccatgtttatggctgcgatggatgcgagtgtcatcaacatggcattgcctgccatcatgaccgaatttcaaacattgccgtctgctgcaggcacagttaacatcggatacttagtcagcttggcggtctgtcttcccgcagccggctggcttggggaccgctttgggacaaagcgtgttttcctagccgcgctttcgatatttacagctgca

SEQ ID NO: 8 - protein, 2385 aa, Bacillus licheniformis
MPECQHNRKPLSGAQAGIWFAQQLDPENPIYNTAEYVEIKGPLDQELFEKALRHVIKEAESFHARFGEDQDGPWQEIVPSTDFPLHYIDVSSETDPEQAAKSWMMDDLARPVDLTRGPLFTEALFKAAQDHYFWYQRTHHIATDGFSFTLIAERLSKIYTALMQNKSIDQSGAFGSLDLILAEETAYRASEQYQEDRRFWLGRFSDEPEVVSLAERAPRTSSSFLRRSEHLPSEDADRLLSAASRMGATWHETVMAAAAIYVHRLTGANDVVLGMPMMCRLGSAALHIPGMVMNLLPLRIGMQPQMSIGELVKQISGEMMKLRRHQHYRHEELRRDLKLLGENQRLFGPQLNLMPFENRLNFAGCQGIVHNLATGPVDDLSINIYGRPDGGGLKVNMDANPAVYHADELEDHGNRFLTLLKTIAVCEQTQPVGTLDILLPEERAQILIEWNQTEHGLPKESLPQRFERMAKECPESPAVVCNDKVLTYSELNQKANQLAHLLIDQGAKPETFIALALPRSAEMVVSMLAVLKAGAAYLPIDPDYPADRIEYMLNDAQPLLVMTSKEAQDTIGSQMPKLILDEQTVMEKMSGCSEENPGEQHSGNQPAYMIYTSGSTGRPKGVVVQAESLFNFLLSMQDMFALNQDDRLLAVTTIAFDISALEIYLPLISGSAVVLAEKETVQDPSELAKMIETYEITIMQATPTLWHALASSAPEKLKGLRALVGGEALQSSLARQLQQLACSLTNLYGPTETTIWSTAAALEGNCTEPPGIGCAIWNTQLYVLDAGLQPVPPGTAGELYVAGTGVARGYLNRHALTAERFIANPYGPPGSRMYRTGDIVRWREDGSLDYIGRADHQVKIRGFRIEMGEIEAVLANHPVVKQAAAIVREDQPGDPRLFAYAVPASGESLDPAELRRFVGETLPDYMIPSAFVILDELPLTPNGKLDRKSLPAPAVSMHTGGREPRTPQEEILCDLFAEVLGVPRVSIDDSFFDLGGHSLLAGRLVGRIREMLGVELGIGRLFDEPTAAGLAKQLDQAQSARPALRKRERRKEIPLSFAQRRLWFLHCLEGPSPTYNIPVVVHLTGDLDQKALAAALGDVATRHEPLRTIFPDQQGTTHQLILEEDQSRPELTVSHVSEHELEKVLAEAVRHRYHLEKEPPFRAQLFVLGPDKFVLLLLLHHMIGDGWSLMPLTRDLETAYNARLQGEAPVWEPLSIQYADYAVWQEYLLGSENNPDSLIAKQLEYWSKALEHLPDQLELPTDHPRPSESSYRSGTIDLSIDEQLHGRLFDLSRSTGVSMFMILQSALAALLTRLGAGHDIPLGSPIAGRNDDALGEIVGLFVNTLVLRTDTSGNPSFRELLNRVRKVNLAAYEHQDLPFERLVEVLNPRRSRARHPLFQIMLAFQNTPEPELDLSGLKSDIEIRSVGAAKFDLTIELREHRKADGTPAGIGGFLEYSTDLFERNTVQTLAERLVRLLDSAADDPDQPIEKLDILLPAERENMLADWSKSSNSIPCSSLPVLFEKQAAKDPEAVAVICENNALTYGELNKRANRLAHLLIAKGVGPEQFAALALPRSLDMVVGLLAVLKAGAAYVPLDPDYPAERIAFMLNDAHPVCIVTSSAVESNLSVPGSVERIVLDDPCIQEELKGCAAANPCDADRTAPLLPLHPAYVIYTSGSTGKPKGVVVPHQNVVRLFGATDQWFHFGADDVWTMFHSYAFDFSVWEIWGALLNGGRLIVVPHTISRSPAEFLNLLVREGVTVLNQTPSAFYQLMQADRDNAETGKLLSLRFIIFGGEALELKRLEDWYERHPDHFPRLINMYGITETTVHVSYISLDQQTAALQANSLIGQGIPDLGVYVLDEYLEPVPPGVTGEMYVSGGGLARGYLGRPDLTADRFVADPFGPPGTRMYRTGDLARRRQDGSLDYMGRADQQIKIRGFRIELGEIEAVLVRHHRVNQAAVVVREGQPGDKRLIAYVVPASEEETDPAELRRFAAGTLPEYMVPSAFVKISELPLTPNGKLDRKALPEPDFAAAVKGRGPRTPQEEILCDLFSEILNAPRVGIDDGFFELGGHSLLAVQLMSRIREALGVELGIGDLLEAPTVSGLAERLESGGRQSALDVMLPLRTGGSQDPLFCVHPAGGLSWCYAGLMTALGKEYPIYGLQARGIARQEELPDTLDDMAADYIRHIRTIQPTGPYRLLGWSLGGNVVHAIATQLQEQGEDISLLVMLDAYPNHFLPIKDAPDEQEALIALLALGGYDPDSLDGAPLNLSSAIDILRRDGSALASLDEAAILNLKETYVNSVRILSEYKPRVFHGDILFFKSTVIPEWFDPIDPESWLPYLNGNIDIHDMDCRHKDLCQPEPLAEIGRRVSEKLDDLKKDTDK

SEQ ID NO: 9 - DNA, 1000 bp, Bacillus licheniformis
  CDS (321)..(998)
ttccgatccatatcatgaaagtgaagggtgggataaatgtatactctacttcgaacacgcttcggaagctaaagcagagcggaaacgaagtccgatctttaaaggcgtacgaacgcagtcgatcttaagcgtcatacgcaaactccgtcgcgactaggttcggaccgcagcaatgagccctggaaggtagctgatgcttcaatttggccggttgtccgcagctgatggggtatgcatgagtcagcgggggttcaaaagtctgaggatgcctttatttaaatgtgtacgcacccgaagaggcggacggggatctgcctgttatggtgtggattcatgggggcgctttttatcgcggcgccggaagtgaaccgctctatgacgggactcagcttgcaaagcagggaaaggtgatcgtggtcaccatcaattatcgcctcggtccgttcggttttttgcatctatcctcaattgatgattcctacagcagcaatcttggcctgctggatcaaatcgcggctctcgagtgggtgaaagacaatatcgctttctttggcggagaccgtcatcacattacggtttttggagagtcggcgggatcgatgagcatcgcttcgcttttggcgatgccgaaagcaaaggggctttttcaacaggccattatggaaagcggggcttccgcaactatgtccgataagcttgcgaaagctgcagcagaaagattcttaaggattctcgatattgatcatcatcatctggagcgccttcatgatgtatctgatcaagaacttcttgaagccgccgatcagctgcgcactttaatgggagaaaatatttttgaattgatttttctgcctgcgcttgacgaaaaaaccttgccgctgaagccggaggtcgccgtcgcaaaaggcgcggcaaaagagatcaatctattaatcggaaccaaacccgtgatgaaggcgtctgttttttcctctgattcgaaacttgctgagagcaatag

SEQ ID NO: 10 - protein, 226 aa, Bacillus licheniformis
MVWIHGGAFYRGAGSEPLYDGTQLAKQGKVIVVTINYRLGPFGFLHLSSIDDSYSSNLGLLDQIAALEWVKDNIAFFGGDRHHITVFGESAGSMSIASLLAMPKAKGLFQQAIMESGASATMSDKLAKAAAERFLRILDIDHHHLERLHDVSDQELLEAADQLRTLMGENIFELIFLPALDEKTLPLKPEVAVAKGAAKEINLLIGTKPVMKASVFSSDSKLAESN

SEQ ID NO: 11 - DNA, 2506 bp, Bacillus licheniformis
  CDS (501)..(2006)
ctctaatagcttcgctttgattgttgtattatttattttgctgatcattgtgggctccgcttttgtaaagtattaattcattcgcgcacgaagccttcttccgaaaaactgcaggttcatatcgaaaggcgctccgcttttcatatgagcctgtttttttgtaatttaaaaagaatttatttccacaataacaaaaaatgtttataataggataggctctgagctgacggctgacggcgctcggaaataaaaactctcccacaaatgaaacttttatacaacattttcgtcttatactgttgttaaggcaattcaataacggaaaggaaaagatcaatgttaaataagctaagaatgttgtatgaaaaaacaacaatgaagtggctgctcaacacttcctattactttctcattctggtgctgctgtttttaatttacggattccatacagcaaatacaggttcatatatttataatgacttttaatggagaagaaaacaatgaaactgattgaaacaattaaaaaatacgcgcaaacacaacccgatacccttgcttttgtgaatgaagaagaaaagctgacatacggagagctttggtcacaatctgagcgccttgccgcaaggattcaaagcgaagctttaacagatgcttctccgatcattgtctacggacatatgaaaccggttatggcggtttccttcctggcttgtgtaaaagccgggcatccgtacattccagttgacgtatcgattccggcagaccggattctgaaaatcatcaacagttcaaaagccgagcttctgctcaataattcaggcacttcggttgacacgggagatgccctcatttccgtagtggaaccgggcgttcttgaaggagacggggtgccggagacagatcccggacgctgggtacatggggaagatactttttacattatctacacatccggaagcacgggaaatccaaaaggcgtgcaaatctcagccgataaccttcagagcttcacagattggatcacgaatgactttccggtggaaagcggccaagtgttcttaaatcaagccccgttttcctttgatttatcggtaatggacctttatccgtgcctgcagtccggaggcaccttatggacggttacgaaagacatgattaatcggccgaaactattgtttgaagcactgaaacagtccaacgtcaacgtctggacatcgacgccttcattcgcacaaatgtgcttgatggatccgtcttattcggaagagctgcttccggagctgagcttgtttatgttctgcggagaaacgcttcctgcgtcagtcgccagacagcttaaagaaagatttccaaaggctcgtgtttttaacacatacggcccgaccgaagcgaccgttgccgtcacttccatcgaagtaacagacgacgtattaaacaagtattcttctcttcccgtcggttctgaaaagcccgaaaccgaaatcgtaatcatcaatgaagacggaaaagccgtccaagacggggaaaaaggcgagatcataattaccggagcaagcgtcagcaaaggctatttaggcgaaaaagcgctgacggaaaaagcgtttttctcttacaacggctcccctgcttaccgcaccggtgacgcaggttacaaagaaaacggccagctcttcttcctcggaagactcgatttccaaatcaaactgcacggctaccggattgagcttgaagaaatcgaatatcagatcaatcagtcccgatatgtgcagtcggcagtcgttattccattctaccgcgaggagaaaatcgagtatctgatcgccatgattgtgcctgccgaacacgatttcgaaaaggaataccagttaaccagcgcgattaagaaagatttgggcagcaagctgccggcctatatgattccgagaaaattcatgtatcaaaaagagattccgatgacagcaaacggtaaaatcgaccgcaagagattaaaagaagaggtaaccgtatgacgccttatggttcatttcttttcttcattatattaggaattttactggcgccgaccatcattctcggactgaacggaaaaagcttccgcttatataatatggcggtttccgttctggttctggcgcttattttttcgaacagcctgcacgggctgatcatgctgtgcctgtttacgctttggcagacggttctgatcaaaggctatatcgcttaccgtctaaaagcgaacagcggcatcgtattttgtctggctgcagcagcttctatcctgcctctggcactgtcaaagctgctgccgtttttcgccgttgacaactgggcaacatttctcggaatctcgtatttaacttttaaaggggttcagctcatcattgaaacccgcgacggtctcattaaaaagcagctcccaatcagcagactgctttactttattctcttttttccaaccatctcgtcaggtccgatcgacaggtaccgccgttttgaaaaagacgat

SEQ ID NO: 12 - protein, 502 aa, Bacillus licheniformis
MKLIETIKKYAQTQPDTLAFVNEEEKLTYGELWSQSERLAARIQSEALTDASPIIVYGHMKPVMAVSFLACVKAGHPYIPVDVSIPADRILKIINSSKAELLLNNSGTSVDTGDALISVVEPGVLEGDGVPETDPGRWVHGEDTFYIIYTSGSTGNPKGVQISADNLQSFTDWITNDFPVESGQVFLNQAPFSFDLSVMDLYPCLQSGGTLWTVTKDMINRPKLLFEALKQSNVNVWTSTPSFAQMCLMDPSYSEELLPELSLFMFCGETLPASVARQLKERFPKARVFNTYGPTEATVAVTSIEVTDDVLNKYSSLPVGSEKPETEIVIINEDGKAVQDGEKGEIIITGASVSKGYLGEKALTEKAFFSYNGSPAYRTGDAGYKENGQLFFLGRLDFQIKLHGYRIELEEIEYQINQSRYVQSAVVIPFYREEKIEYLIAMIVPAEHDFEKEYQLTSAIKKDLGSKLPAYMIPRKFMYQKEIPMTANGKIDRKRLKEEVTV

SEQ ID NO: 13 - DNA, 11740 bp, Bacillus licheniformis
  CDS (501)..(11240)
tttttttaatattttaatatactctttatctaagatttggaattttttgcgggtttaaacgatttttttcgttttcccgcatatttttttgtcaatttcgggatcgcgcattaacattcatgtacaacattcggtatcccgcatgaaacttttcacctatttttcggtgaaaaaaacaaaattttcatttaaagtgaactgtgagcagaaaatgaatttatatcaagaaaagcagataaaggcaaacctgcggaaacgcagggacgcaaagccatggcctaaggtgctgacggtgctacggttgacaggttgccgaataaacagggagttcgcccgtttttattcgggcgggctcttttctttttatttccaatataatgttttattggaaacgacaaatctgtgacagcgtttttcgctcatcgcaaaaccgcaacattgcattgcggcttggctgttcgcatcgtcatacataacaagagataaaggaggaataaagaatgggaaacacattttacccgctgacacatgctcaaaggcgaatttggtacacggaaaagttctatcccggtacaagtgtttcaaatttgagcggctttggaaaattaaaatctgcatccggaatcgattccgggttattgactgaagcgattcgcaagtttgttcgtacgaatgacacgatgcgattcaggctgatgtttgaaggagaggatgagccgaagcagtatatagcggaagacgaacctttccaaattgaatattttgatgcttcagaaagcggaggagcagacggcgttcttaaatggggacaagctgaggcgcgccggcccctgcctttgtatgacagtccgctgtttaaatttgccgttgttcgcattagtgaagaagaaagctggttttttgcaaaagttcatcatatcatttccgacggcatttctatgacgatcctaggcaaccggattacagatatttatttgaagcttgccaaaggggaaaccgaccttgaacctgtacagtcatcatttacggaacatatccagagcgaattggagtatgaaaattcgaagcgtttccagaaggataaagcatattggaacgcacaatatgaagcgattccggagcccgtatcgttaaaagcgagcgatacgtatcagatccaattggatgcagcccgcttttcaaaagagatttcccctgacctgtataaaaaaattcaaacgtattgtaatgaacacaacatcagtgtactctccctgttcctatccattctgcacatctatatgcaccgggtaaccggacaaaaagacgtcgttctcggaacatttatgggtaaccggacaaatgcaaaagaaaaacagatgctcgggatgtttgtgtcgacgattccgatgaaagcctccattgaagtacaccaagactttagtgcatttgtgcaggaaagaatgaaggaccagctgaaaatcattcggcatcaaaagtatccgtacaatctgctgattaatgatttgcgggaaagacagccgcatgtcagcaaattgttcgccgtctcactggaatatcaggttatgcaatggcagcaaaaagaaacggtgtcgtttcttaccgagccgattttcagcggcagtgaagtaaacgatatttccattcatgtgaaagagagatgggataccgggacgcttgcgattgattttgattacagggaggagctttttacttcggaagaaatggccgtgttatatgaacggctgatgacgctgcttgaagatgccctgctgtctccgcaaaaaacaatcgctgaactggagattgttcccgcttttgaaaaagagcggctgttaaagcgtgcatcaagccaaaccattgcatatgacaagaacatgaccctgcacggtttatttgaacaaaaagcagccgatcatcccgaaaaaacagcggttgtatatgaaggacaaaaactttcgtaccgggagttgaatgaacaatcgagccgcttggcaatggcgcttcggagaagaggcatcgggcctgatgctccggctgcgatagtgatggagcggtcagagcgggtcatcacggcgatgatgggcgttttgaaagccggcggagcttatgtaccgatcgatcctggatttccggaagaaagaatccgttttatgctggaggattcaaaggcgaaagcggttatcacagacagcgggctgacttttgaaacggctgaaacggtgcagttcagcgaagcgctttctgaatcgcgggaaaacggctatccgtcttcagcggcaggcgccgggcaccttgcatacatcatttatacttccggtacaacgggccgtccaaaaggcgtcatgattgaacacaggcaggtccaccaccttgtcagaggacttcaacaggccgttggtacatacgatcaggatgatttgaaactggcgcttctggctccatttcattttgatgcttccgttcagcagatttttacgtctctattgctcgggcaaacgttgtacatcgtaccgaaaaagacggtttccgacggacgcgctttatcggattattaccgccggcatcaaatcgatgtgacagacggtacaccggcacatttgcagcttttggctgcggctgatgatctttcaggcgtcaaattgaggcatatgcttgttggaggagaggctttatcaagggttgcgacggaaaggctgcttcagttatttgcagaaacagctgaatccgtgcctgctgttacgaatgtttacggaccgacggaaacttgtgtggatgcctcttcttttacgattacaaaccgcacagatcttcaatacgatacggcctatgtaccgattggccggccgataggaaataaccgtttttatattttggatgaaaacggcgcactccttcctgatggcgtcgaaggggaattgtatattgcaggagacggagtcgggcgaggctatttgaacttgcctgacatgaccagagacaggttcttgaaagacccgtttgtatccggaggtctcatgtatcgaacaggggatactgcacgttggcttcctgacggtacagttgattttatcggccgaagagacgatcaagtgaaaatccgcggcttccggatcgagcttggcgagatagagtcggtgctgcaaggagcccctgctgtcgaaaaagcagttgtcctcgcgcggcatgaaaccggcggcagccttgaggtttgcgcttatgtcgttccgaaacaaggcggaaagattcacattcagggactgagggagcacttatccaaacacctgcctgattatatgatcccttcctgctttgtcgagcttaatgaaataccgctgacggcaagcggaaaagtcgaccggaaagcgctgcttcgccatgaggtgtctgtcagcgggactgcagaatatgcggcgccgagaaatgaatgtgaagaaaaaatggtgggtatatggcaggaagtgctcggagctgaacaagtcggaatccacgaccagttttttgatctgggcggccactctctcaaagcgatgaccatgctggcaaaaattcataaagctttcggagtcgaagttccgttgcaagtcctatttgaaaagccgacagtagcggcgctgagcggctttgtttctgaagccgagaaagacggattcgcggtaattgaaccggcgccggaaagtgacgattatcctctttcattggcgcagcagcgaatctatatcgtcagccagctggagggtgtcggctacaatatgccggccgcggcgatgcttgaaggcactcttgactctgggcgccttgaagctgcttttcaaaaactgattgataggcatgaggcgctgcgtacttcgtttacagttgtggatggcgagccgaggcagaccgttcatcaaagggttcagttcaagattgagaaggttaaggcggaaggcaagccgatcgaacaaatcgcgaaatcatttgtccgccgctttgatctcgccaaagctccgctgatgagagcgggtctcgtcagccttgcagacggcagacatctgctgttgttcgatatgcatcatctcgtatctgacggcgtgtcgatttcgatcatattgaatgaactggccgcgctgtataacggggaggaattgccggagctgcgtttgcattacaaagattatgccttctggcaaagggcgcaggcacaagaaggctttcaaaaagaagaggcctattgggaaagcatgtttgccggcgagcttcccgtactgcagctgatgacggacgagccgaggccgcctgttcaaagctttgaaggagacagggtatccgcagtcctgccaaaagacctgaaagaaaaattagccgtgctggcagagcaaaacggcgcgacattgtacatggtcatgctgtctgcgtacaacatgcttcttgcaaagtactccgggcaggaagatgtgatcgtcgggacgccggcggccggccgcagacattccgatctggaaggaatcatcggcatgtttgtcaatacgctggcgatccgttcaaaagtggacccgggcagaacgttcgcagacttcctgaatgatgtaaagaaaacggtaatcgatgcttttgagcatcaggattatccgtttgagcggcttgcggagaagtttggcgcatcgcgcgatttaagccgtcatcccatctttgacacgatgtttattttgcagaatgcatgggaagacattccgctgctcggcgatttgcacctctcgatttatgaaacgaacttcaatattgcaaaattcgacttgacgctgcaggcaaaagaagagcagggagaattgatactcgatttggattacagcacgaagctgtttaaaaaagatacggctgaaaggatgctgaaagcatacttgaaccttctggaggacatggcggctgatccgatgctcaggatcggcgagtacagccttttgacagaagaagaaacgaatcgccagctcgtcgcgttcaaccccgcgacaagcgattatccgcgggagaaaacgatcgttcagctattcgaggaacaggctgcagaaagaggcggttatcccgccctgcaatttgaagacaaagtatggtcatatgatgagctcaatcggaaagcgaaccaattggcaaggcgcctccgcgaaagtggcgtgcaagccggcacgacggcggccattcttacggccaggtcggctgaaatggtcatcggcattctcgctgtgctgaaagccggcggggcttatgtgccgatcgatcccgatcatccggaaaagcgtgtgcaacatttcttcaaggacagcggagcggcggttcttttaacgcaaaaggcgatgaagccgctcgctgaggcggcggaatttggcggcgacatcctatttgttgaggatgaaaacctttatatgggagatgcgtctgatttgcgccttccgatttctccggaagcaatggcaaatctaacgtacacatctggaacgacagggacgccgaagggcaatatggtctctcaccggaatattttgcgtactgtaaaaaacgcgaattacttggaagtgatggaatccgatatcgttctttcgatttcaaattatgtgttcgacgcctttatgtttgatgtgttcggttcattgctgaacggcgccaagctggtgatagctccaaaagacacgattcttgatatgagccggctcgcacatgtactcgaaaaagaaaagattacgattttaatgatcacgaccgcattattcaatctgctgacagacatgcggccggacagtttgaagggactccgcagggttctgttcggcggcgaacgtgcatctgtcgatcatgtcagaagagccctgaagacagttggcagagggcggctgcttcatatgtacggtccgtctgagagcacggtctttacaacctaccatccagtcaatgaagtgccggacgatgcgcaggctataccgatcggaaagcctgtcagcaatacggaggttctgattcttgattcattcggcaatgtacagccggcaggggttgcgggagaattatgcgtcggcggagacggccttgtccgcggctattttaaccgccctgaactgacggctgaaaagtttacggcgcatccgttcaaaaccggcgaaaagatttaccggacgggtgatatggcgagatggcttgccgacggatgcctcgaatttatcggtcggattgatcatcaggtcaaaattcgcggacagagaatcgagctgggcgaaatcgagcatcatctcctaacgcatgacatggttcaggaagccgctgttcttgctgttgacaccggggcgggagatcaaatgatctgtgcatactttacggctgatcaggagctttcgagccaagagcttcgccgacatgcggcagaaggtctgccaggctatatgatcccgtctgtgtttatgcagcttgatgagcttccgctcacaggcaatggaaagatcgacagaagggcgctgcctgaaccggatatcgcgcaggccgcgcaaaaggaatatacggcgccaaggagcggcacagaagcgcagcttgccgatctttggcaggaggtgctcaatgtaccgaaaatcggtgtgcatgacaatttttttgaactcggcggacactctttactcggaatgacgctcatcgcccgcatccagcaagagatgaatgttgacctgcagctgaaagacttgttccaggcgccgacaatcgaaagcctggcccaggcggcggcaaaatctgagaaaaaatctgccgtttatattgaagcggcgccggaccgggaaacgtaccccgtttcgtcggcgcaaaaacggctgtacgttcttcagcagattgaaggcgctgagaaaagctataacatgccggccgttctccaacttgaaggaaagcttgacctgaagcgcctggagtctgccgcgcaaatgctgatcaagcgccatgaagcgttccgcacgacgtttgaaatcaggaacggcgaacctgttcagcggatttgggaagctgcggaattgaccattgatgtcatcgatgcagatgaacaagaagcggaaaagctgattaaagagttcatcaggccgtttgatttaacaaaggctccgttatttagaatgagcatcatacgtgtcacggaagaaaaacatcttttgcttgtcgatatgcatcatatcatttcagacggcgcttccgtcagcgtgctgatcgatgagatgacaaggctttatgcgggagaggcgcttgaaccgctgaggattcagtataaagactatgctgtatggcagcagcacttgctcactgaacggcacaaaatgcaggaggaatactggctcaaggagcttgacggcgaactgcctgttttgacgctcccgacagactatccgcgtccttctgttcaaacgtttgaaggaagccgtatcagcttttctctgaaaccggagcttgtccagcagctcagaagactggccaaggaaacggaaagcactttatacatggtgctcgctgcctcatacagtacgtttttatcgaaactgagtggacagagcgaggtcattttcggctctccggctgccgggaggccgcatgcagatttgagccgtatcatcggcatgtttgtcaatacgcttgcgataagaacgcgtcctgaaggagacaagccgttttctgcattccttgaggaagtaaaagaaacgacgctcggcgcatttgagcatcaagattacccgtttgaagaactgatcgagaagctgaacatccagcgcgacatgagccgaaacccgttatttgatgccgtcttttcaatgcaaaatgccgatttgaaagacttgtcaatggaaggagtcacacttaaaccgtatgattttgcccatcagactgcgaaatttgatctgacgttgacagcggcggaagaagacggacttctcgtttttgaaatggaatacaatacagatctctttaagcgcgaatcgatcaaacgctggagcgggtactgggtcaacctgctcgaagcggttgccgaaaccccggatgcaagtctttcagacctttcgcttctggatgaagcggaaaaacggcggatcgtccaaaagtggaatgagacgaagctcgctgtgccgcaggacaaaacggtccatgagctgttcgaagcgcaagtcctccgcactcctgaccgcggggcagcggtgtacaacggcgtgaagtggacgtacaaagagctgaacgcgcgcgccaaccgtctggcccgcctgctgattgaaaaaggagccggtcctgagcagcgggtcggaatcatggtgaagccttcgctggaaatggcggccggcgtacttgcgattttgaaagcgggagcggcctatgtaccgatcgatcccggctatccggccgagcggatcggatacgtactgaaagacagcggtgcagaactgcttctgacgcaaacaaacttagccgtacctgaagaattcagcggtgaaacgcttcttctcgattcgatgctgagcgaagaaatcacaaatgatgatgaagtgaacccgcaggcaggtacacagccaaacaacctggcatacttgatttacacatcgggcacgacaggccagccgaaaggcgtcatggtcgaacatcaatcgctcgtgaacttgtgctattggcataacgatgcattcgcggtcaccgaacaggacaaaagcgcgaaatatgccggcttcggctttgacgcttccgtctgggagatgttcccgtactggatcgccggagcggagcttcatatcattgatgaagcgatccggatggacattacgcgtttgaatgaatatttcgaagagaacggaataacgatcacgttcctgccgacacagctgtgcgaacagttcatggagctcgacaaccagtcgctgcgcgtgctgcttacgggcggagacaagctgaagcggatcgaaaaacggaactacacgctcgtcaacaactatgggccgacggaaaacacagtcgttgcgacgagcacagcaatcgatcctgatgaaggcatgctttcgatcggaaagccgattgctaatacgcgggcgtatgtgcttggacaaaacaacgaagttcagcctgtcggcgtcgctggcgagctgtgcatcgccggccgcgggcttgcgcgcggctatctgaacaagccggaggaaacggcgaagcgttttacggaagacccgtttgtgccgggcgagcgcatgtaccggacaggggatgcggtcaaatggctggaagacgggcggcttgagtacatcggccggatcgaccagcaggtgaaaatccgcggcttccggatcgagctttctgaaattgaagtccagctcgcgcggctctctgaagttcaggaagcagtcgtcaccgacatcgaagacgcctacggcaacaaagcgctgtgcggctatgtcgttgcggacgaacagcttgatacagaaagcctcgcccgtaagctggggcagacgcttccggactacatggtaccggcgtattgggtgcagcttgatgagcttccggttacggcgaacggaaaagtcgaccgaagggcgctgcctcagcctgacgtcgaagcgcagacagccgaatacaaagcgccgcgcacagaaacggaacagctgttggcggacatttggcaggatgttctcggcatagaccgaatcggagtcacggacaatttcttcgcgctcggcggggactcgatcaaaggaattcaaatggcaagccgccttcagcagcacggttggaagctggagatgaaggacttgtttcagcacccgacgatcggggagctcagtgcttatgttcaagcggctgacgatcagccgatcgatcagaaccctgtcgaaggcgaagtcacactgacgccgatccagcgctggttctttgagcgcaaatttacgaacgagcatcattggaaccaatcagttatgcttcacgccccttcagggtttgacccggaagcggcaggcaaggctttaaccaagatgatcgaacaccatgatgcacttcgcatggtttatcagcgaaatggagacgggattgttcagtacaacagagggctggaagaaacagcggttcgtcctgaagtcatccgcttcaatgaatcgggtgctgaacttgaagctgccgtgttaaaggaatcaaaccggattcaaagcagtatcaatttaaccgaaggcccgcttttgaaagcggcgatctttgagacggatcaaggcgatcatcttctcatcgtcattcatcatctcgttgtggacggaatttcatggagaatcctccttgaggatttcgccgccgggtacgcccaagctgaaaaaggggagccgatcattcttcaagaaaagacgcactcttttgctgaatatgcggctcgcctgaaagaatacgcaaggtcaaaagctttcgcgaaagaaatcggctattggcaggaagtcgagaaggcggagacggcatcgctccctaaagatgatgaagcagaggacaaacggatgcaccatacgaaaacggctgagttttctctctcgaaagaagaaacagaacagttgatgacaaaagttcatgaagcatataacacggaaatgaacgacattttgcttacggcgttgggcttggcgctgaaagagtggaccgggcaggaagactttatcatctgcttggagggacacggccgggaggacatcatggaaggcttgaatatttcccggacggtcggctggtttacaagccagttcccggcgctgattcaactgcgccattccgaagatatcggctatcaaatcaaacagatcaaagaagagttgcgccacattccgaataaaggaatcggctatggcatttatcgctatttgaccgaagaaggcaaaaaggcgcagccgatcaagcatgatatcagctttaactatttgggccagtttgcagagatggcggattcgggtctgttcacgagatcagctttgccgtccggcgatccgctcagcccggaaacggaaaaaccgaatgcgctagacatcgtcggctatatcgaaaacggcattttgacaatgtcaattgcctatcattcactcgaatacaaagaaagcaccgtcgccgctgtcgccgcaagctttaaaacttatttgctgcaattgatcgatcattgcttagagctggatggaggcgaactgaccccgagcgatcttggagatgacgaactaacgcttgaagaattagataaactgatggaaattttctaacaaaaaaacgaggtgccttatggggaaacaaaagattcaaaaggtgtatcctttgacaccgatgcaggaaggaatgctgtaccacgcaatgcttgatccggaatcttcctcttacttcacacagctggagcttgtcatagacggggaatttgatttggggatctttgaaaaaagcgtgaatcagctcgtgcgttcatacgatattctcagaacggtttttgttcatcagcaattgcaaaaaccgcgccaggttgtcctggctgaacggcaggcaatagtggaatttgaggatcttgcggacttggatgaggaaaggcaaaaaacccgcattgaccggtataaacaagaggtacaggcggccgggtttaatttggcgaaagacatgctgtttaaaacggcggtctttcggcttgaccggaaaaaataccatctcgtctggagcaaccatcatatcgtgatggacggctggagcatggggatattgatgaaacgccttt

SEQ ID NO: 14 - protein, 3580 aa, Bacillus licheniformis
MGNTFYPLTHAQRRIWYTEKFYPGTSVSNLSGFGKLKSASGIDSGLLTEAIRKFVRTNDTMRFRLMFEGEDEPKQYIAEDEPFQIEYFDASESGGADGVLKWGQAEARRPLPLYDSPLFKFAVVRISEEESWFFAKVHHIISDGISMTILGNRITDIYLKLAKGETDLEPVQSSFTEHIQSELEYENSKRFQKDKAYWNAQYEAIPEPVSLKASDTYQIQLDAARFSKEISPDLYKKIQTYCNEHNISVLSLFLSILHIYMHRVTGQKDVVLGTFMGNRTNAKEKQMLGMFVSTIPMKASIEVHQDFSAFVQERMKDQLKIIRHQKYPYNLLINDLRERQPHVSKLFAVSLEYQVMQWQQKETVSFLTEPIFSGSEVNDISIHVKERWDTGTLAIDFDYREELFTSEEMAVLYERLMTLLEDALLSPQKTIAELEIVPAFEKERLLKRASSQTIAYDKNMTLHGLFEQKAADHPEKTAVVYEGQKLSYRELNEQSSRLAMALRRRGIGPDAPAAIVMERSERVITAMMGVLKAGGAYVPIDPGFPEERIRFMLEDSKAKAVITDSGLTFETAETVQFSEALSESRENGYPSSAAGAGHLAYIIYTSGTTGRPKGVMIEHRQVHHLVRGLQQAVGTYDQDDLKLALLAPFHFDASVQQIFTSLLLGQTLYIVPKKTVSDGRALSDYYRRHQIDVTDGTPAHLQLLAAADDLSGVKLRHMLVGGEALSRVATERLLQLFAETAESVPAVTNVYGPTETCVDASSFTITNRTDLQYDTAYVPIGRPIGNNRFYILDENGALLPDGVEGELYIAGDGVGRGYLNLPDMTRDRFLKDPFVSGGLMYRTGDTARWLPDGTVDFIGRRDDQVKIRGFRIELGEIESVLQGAPAVEKAVVLARHETGGSLEVCAYVVPKQGGKIHIQGLREHLSKHLPDYMIPSCFVELNEIPLTASGKVDRKALLRHEVSVSGTAEYAAPRNECEEKMVGIWQEVLGAEQVGIHDQFFDLGGHSLKAMTMLAKIHKAFGVEVPLQVLFEKPTVAALSGFVSEAEKDGFAVIEPAPESDDYPLSLAQQRIYIVSQLEGVGYNMPAAAMLEGTLDSGRLEAAFQKLIDRHEALRTSFTVVDGEPRQTVHQRVQFKIEKVKAEGKPIEQIAKSFVRRFDLAKAPLMRAGLVSLADGRHLLLFDMHHLVSDGVSISIILNELAALYNGEELPELRLHYKDYAFWQRAQAQEGFQKEEAYWESMFAGELPVLQLMTDEPRPPVQSFEGDRVSAVLPKDLKEKLAVLAEQNGATLYMVMLSAYNMLLAKYSGQEDVIVGTPAAGRRHSDLEGIIGMFVNTLAIRSKVDPGRTFADFLNDVKKTVIDAFEHQDYPFERLAEKFGASRDLSRHPIFDTMFILQNAWEDIPLLGDLHLSIYETNFNIAKFDLTLQAKEEQGELILDLDYSTKLFKKDTAERMLKAYLNLLEDMAADPMLRIGEYSLLTEEETNRQLVAFNPATSDYPREKTIVQLFEEQAAERGGYPALQFEDKVWSYDELNRKANQLARRLRESGVQAGTTAAILTARSAEMVIGILAVLKAGGAYVPIDPDHPEKRVQHFFKDSGAAVLLTQKAMKPLAEAAEFGGDILFVEDENLYMGDASDLRLPISPEAMANLTYTSGTTGTPKGNMVSHRNILRTVKNANYLEVMESDIVLSISNYVFDAFMFDVFGSLLNGAKLVIAPKDTILDMSRLAHVLEKEKITILMITTALFNLLTDMRPDSLKGLRRVLFGGERASVDHVRRALKTVGRGRLLHMYGPSESTVFTTYHPVNEVPDDAQAIPIGKPVSNTEVLILDSFGNVQPAGVAGELCVGGDGLVRGYFNRPELTAEKFTAHPFKTGEKIYRTGDMARWLADGCLEFIGRIDHQVKIRGQRIELGEIEHHLLTHDMVQEAAVLAVDTGAGDQMICAYFTADQELSSQELRRHAAEGLPGYMIPSVFMQLDELPLTGNGKIDRRALPEPDIAQAAQKEYTAPRSGTEAQLADLWQEVLNVPKIGVHDNFFELGGHSLLGMTLIARIQQEMNVDLQLKDLFQAPTIESLAQAAAKSEKKSAVYIEAAPDRETYPVSSAQKRLYVLQQIEGAEKSYNMPAVLQLEGKLDLKRLESAAQMLIKRHEAFRTTFEIRNGEPVQRIWEAAELTIDVIDADEQEAEKLIKEFIRPFDLTKAPLFRMSIIRVTEEKHLLLVDMHHIISDGASVSVLIDEMTRLYAGEALEPLRIQYKDYAVWQQHLLTERHKMQEEYWLKELDGELPVLTLPTDYPRPSVQTFEGSRISFSLKPELVQQLRRLAKETESTLYMVLAASYSTFLSKLSGQSEVIFGSPAAGRPHADLSRIIGMFVNTLAIRTRPEGDKPFSAFLEEVKETTLGAFEHQDYPFEELIEKLNIQRDMSRNPLFDAVFSMQNADLKDLSMEGVTLKPYDFAHQTAKFDLTLTAAEEDGLLVFEMEYNTDLFKRESIKRWSGYWVNLLEAVAETPDASLSDLSLLDEAEKRRIVQKWNETKLAVPQDKTVHELFEAQVLRTPDRGAAVYNGVKWTYKELNARANRLARLLIEKGAGPEQRVGIMVKPSLEMAAGVLAILKAGAAYVPIDPGYPAERIGYVLKDSGAELLLTQTNLAVPEEFSGETLLLDSMLSEEITNDDEVNPQAGTQPNNLAYLIYTSGTTGQPKGVMVEHQSLVNLCYWHNDAFAVTEQDKSAKYAGFGFDASVWEMFPYWIAGAELHIIDEAIRMDITRLNEYFEENGITITFLPTQLCEQFMELDNQSLRVLLTGGDKLKRIEKRNYTLVNNYGPTENTVVATSTAIDPDEGMLSIGKPIANTRAYVLGQNNEVQPVGVAGELCIAGRGLARGYLNKPEETAKRFTEDPFVPGERMYRTGDAVKWLEDGRLEYIGRIDQQVKIRGFRIELSEIEVQLARLSEVQEAVVTDIEDAYGNKALCGYVVADEQLDTESLARKLGQTLPDYMVPAYWVQLDELPVTANGKVDRRALPQPDVEAQTAEYKAPRTETEQLLADIWQDVLGIDRIGVTDNFFALGGDSIKGIQMASRLQQHGWKLEMKDLFQHPTIGELSAYVQAADDQPIDQNPVEGEVTLTPIQRWFFERKFTNEHHWNQSVMLHAPSGFDPEAAGKALTKMIEHHDALRMVYQRNGDGIVQYNRGLEETAVRPEVIRFNESGAELEAAVLKESNRIQSSINLTEGPLLKAAIFETDQGDHLLIVIHHLVVDGISWRILLEDFAAGYAQAEKGEPIILQEKTHSFAEYAARLKEYARSKAFAKEIGYWQEVEKAETASLPKDDEAEDKRMHHTKTAEFSLSKEETEQLMTKVHEAYNTEMNDILLTALGLALKEWTGQEDFIICLEGHGREDIMEGLNISRTVGWFTSQFPALIQLRHSEDIGYQIKQIKEELRHIPNKGIGYGIYRYLTEEGKKAQPIKHDISFNYLGQFAEMADSGLFTRSALPSGDPLSPETEKPNALDIVGYIENGILTMSIAYHSLEYKESTVAAVAASFKTYLLQLIDHCLELDGGELTPSDLGDDELTLEELDKLMEIF

SEQ ID NO: 15 - DNA, 1738 bp, Bacillus licheniformis
  CDS (501)..(1238)
aaggttatgtataagcaacatattaccacaatttgcgcagaaacacgaaaaacaggatgaacggctccaacgttcaccctgcctgaagaaggttacgctttaatgaagaaacgttaactgccttttgcaagcacccatttttcgtcaaaatgaagacttgtaacgcctatacatttcctgcgacaaacacctcatttgatcgtcacaccttttttatatgacgaagacgtcaaataaagcaggtgctttctcaattcagtggatgaaagaaatttctaagcttgacatgtttcatactcaggactcttgccggattttgtctttgacaaacacctcattcgccagagttgataagctttcccttcttcaagaattagaatgtcctaaggcgctttcgattattcgttttatttttacggccgaaaagcgggagggaaaacgtttcaaatacattcttgtttttggtactattaagaaaaatgcaggaaaggaacgaacgtatgtcactactgactgttaaagagttaactggagggtatacaagaaacccggtgctgcgcgatgtttcatttgcgattgaacgggagcagatcgtcgggctgatcggtttgaacggagcggggaaaagcacgacgatccggcatatcatcggcttaatggaaccgcacaaaggatctgttgaattgaacggcaaaacgttcatggacgatccggaaagctaccgttcccaatttacgtttattccggaaacgccgattttatatgaagagctgacattgaaggagcatttggagctgactgcgatggcgtacgggctttcgaaggaaacgttcgagaaaaggctgccgccgcttttgaaggaattcagaatggagaagcgtctgaaatggttcccggcccatttttcaaaaggaatgaagcagaaggttatgatcatgtgcgctttccttgtcgaacctgacctgtacattatagacgaaccgtttctcggtcttgatccgctggcgatcaatgcccttctcgagcggatgaatgaagcgaagaaaaacggctcggccgttttgatgtcgacgcatattttggcaacggcagagcgatattgcgactccttcatcattctccataacggcgaagtgcgggcgaaaggaacgcttgaagagctcagagaacagttttcgatggaagaagcgagcctcgatgacctgtatcttgaactgacgaaggaagagagctatgaatagcattctggggatttggaagacaagaattgaggagtatgtcaaagaaacaagatcatatttgaaatacatgctgaatgaccaccttgtcatcgtcatgatttttttcctcgctggagctgcgagctggtacagcaaatggctgaaagagatgcccgaaggttttccagcctattgggtgatggccgtcatcttttcgctcattttgacaggctcgtacgtccgcacgctgattaaagaagctgaccttgtctttttgctgccgcttgaagcaaagatggggccgtattttcagcaggctttccggtttagttttttgacgcagctgtttcctctggccgccgcggccctcgctttggcgccgctgtattttgaagcaagcggcaaggggttttccgcgtacctcgtcttgctgattcaactgctcgtcttaaaaggatggaacacggctatgcaatggcggatgacgtttttcggcgaaaaaaacatgagggttaccg

SEQ ID NO: 16 - protein, 246 aa, Bacillus licheniformis
MSLLTVKELTGGYTRNPVLRDVSFAIEREQIVGLIGLNGAGKSTTIRHIIGLMEPHKGSVELNGKTFMDDPESYRSQFTFIPETPILYEELTLKEHLELTAMAYGLSKETFEKRLPPLLKEFRMEKRLKWFPAHFSKGMKQKVMIMCAFLVEPDLYIIDEPFLGLDPLAINALLERMNEAKKNGSAVLMSTHILATAERYCDSFIILHNGEVRAKGTLEELREQFSMEEASLDDLYLELTKEESYE

SEQ ID NO: 17 - DNA, 4846 bp, Bacillus licheniformis
  CDS (501)..(4346)
ctgacaaaggaatcggctacggcatccttcgctacttaacagaccggaaaaaagccggtgcaattttctcgatcaagcctgacatcagctttaactacctgggacagatcgacagggaggtgcagaccggcttctttgggccttctccttatgatatgggaagacaggtctctgaaaggtctgaagcactctatgcactgagcttcagcggcatcatcagcaaggaaaaattcatcttgtcatgttcattcaatacagaggaatatgacaggcagaccatacaggagctcatggacagattcaaagctttcctgacagcattgatcgaccattgtacagctaaagaagaacgcgaatttacaccgagtgatttttctgccggcgatcttgagatggaagaaatgggcgacctgtttgacgttcttgaagaaaacttgaaataaccaccgaggacttgcccgcttgtcaagcgggcgagcctatgaaagggggatacgatcttgagccagtttaaaaaagaacatgtccaagacatatactatttgtcaccgatgcaggaagggatgctgtttcatacgctgctgcatccggggcaaggcttttatattgaacaaatcagcatgcaggtgaaaggctcttttcaaaaggatgtattggaaaaaagcatgaatgtcatcatcggcaggtatgatatattccgaaccgttttcgttcatgaaaaaatgaaacgtccggttcaagtcgttttaaaggaaaggtcatttcaggcagaagaaattgatctatcaggactttctgaagcagaacaaaatgagagaattgaagattataaacgaaaagataaagaaaaaggatttaacctctcaaaagacattccgatgagaacggcggtttttaagaaggggcaggatcgttacgaatgggtgtggagctatcatcatattctgcttgacggatggtgcttcggcattgtcgtacaggagctgttcgaagtatacaacgcgctccgtgaaaacaggccatacagcctcgggcctgtcagaccttacaaagaatatattaaatggctcgaaagccaggacaaacaaaaatcgctggtctactgggaacatcatttagccggattcgaagggcagacgacattcagcgaacagcggaaaaacccggagcaagccggcaatcagccggaagaactgctgtttacacttccaaaagaagatacagaagcgttcacacagctagcaaaagcgcatcataccacgttgagcacggcgcttcaggcggtgtggtcggtgctgctcagccgctatcagcgatcccgcgatctgatcttcggtacagttgtttcaggccgccccgctgacattcaaggtgtcgagcacatggtcggtttgtttatcaatgtcgtcccgaaacgggcgacctttggcccgcaaacgacctttacggagctggtcgctgatttgcagaagcaggcgctcgaagctgaaccgcatcaatatgtgccgctctatgaaattcaaagtcatatagcagcgccgaacttaatcgaccatattatcgtttttgaaaattacccgctgcaagaagccaataaacagcagaaagaaaaaaatctcggcttcacaatggacgagatcagcgtttttgaaaaatcgaattatgatctgaatttgcttgcttcaccgggtgaagaaatgatgctgaaacttgcatacaaccggaatatattcgaaccatctttcatcttgcggttaaaagaacagcttttgacagccattcgcgaggtcatcagagatccggagcagccgcttgacgacatttgtgtcgtttccaaaaaggaaaaagagcgacttctccatgaatttaacggtcaagtccaacggaagcaaatcggcctgacgattccccaatggtttgagcaatacgctgaagcgctcccggaaaagcccgcggtatcagcggccgggcggacgctgacatatcgggaattgaacgaaaaggcgaacaaggtcgcgcaccttttgcgcaaaaagaacgtcggccgcggcgagcccgtcgcccttttgttccgccgttcgccggaaatggtcatcgcgatactcgccgtcttaaaagcagggggcgcctatctgccgattgacccggaatatcctgaagcacggattcagtatatgctggaagacagcggggctgtttgcatgctgacacaggaagagcttgccggccgggcggcgtcgctttcttttcataagcatacgatcctgatcgacgatcctgctgtgtctgcagaaagcgggagaaacctcgaaatcgccgccgagccggacgaccttgcttatattatgtatacgtctggcacaacgggaaaaccgaaaggcaatctgacgacacatgccaatattacaagggttgtaaaagagacaaactacatcagcctttccgagaaagacaccttgctgtccctttcaaactatgcgtttgacggcttcacatttgatctgtacggcgcgctgttaaacggagcaaagctggtggtggccgatcaggcgacgattctccatatcggcaagctgacagaagcgattcagaaggaaaacatcacagtcatgtttgtgacaacggcgctgttcaacttgctggttgatgcgggaacagggtggatgaaagggattcgcaaagtcctgttcggcggagagcgctcatctgtcagccatgtcaagaaagcatttgctgccatgggaccggacaggatcatccatgtatacgggccgacggaaacgaccgtcttcgcgacgttctatccggtcaaccgtatggaagacagcgcggtttccattccgatcggcaagccgataaacgaaacgaatgcatacatcctgacggaaaataaccggcttcagccgattggagcggtgggtgaattgtgtctgagcggtacaggagtatcgcgcggctatttgaacaggccggaactgactgcggaaaaattcgcgccgcatccgtttaacagcggtgaaacgatgtacaggacgggagatctggcccgctggctgccggacggcaatatcgacttcatcgggcggatcgatgatcaagtgaaaatccgcggacaccggattgagctcggtgaaatcgaagagcagctgatgcgctgccaaggcgtaaaggaagccgttgtaaccgccaggaaaagcgggaacggggatgcggcgctcaccgcttatgtcgtcccggtgcagggaactgaggttgcaaacgaggaggttcgccgccaattggcgcggaggctaccggcttacatggtgccggctgcgtatatcatgcttgaagagctgccgcttaccgctaacggaaaagtcaaccgacgccttctccctgaagccgatggccggccgaattcaacagagcgccgtgcaccccgcaatgctacggaagaaaagcttgcagtgatttggtctgaggttctcggcagacagcagatcggaatagacgagaacttttttgaaatcgggggacattcattgaaagccatggccgtcgcctcccggatgctgaaagatctcgcaatcgatgttccggtgaatgtgttgtttgaaatgcccacaatcgagggcctcgcggcttatatcgaccaaggcggccatgtggatgaagtgaaaaaaaccgtcttcaatcaggcaagcacccgccatttgttcgctttcccgcctgtcctcggatacggcatcatgtacggcaagctggccgaacaaatgccggaatacaggattcacgcgttcgactttatagaaagcgatgaccgcgctgtgcgctatgcagaagccatctccgaccttcagcctgaagggccgctgacattgatcggctattcagccggctgcggacttgcctttgaagtggcgcaagcgctcgaaaagaacggacggaaggtcgaaaagctgctgatgatcgactcatatatgaagaacgatgtcagcgatttagaagggcggtcaattgaagccgatacggccgccttgatggaagccaacaaggacaatgaatatatgaaaatcaagacggttgccgatggcatcgctaaaaagatgacagcctattattcttactttgtcaatcttgtccatcaaggaacggtcagctcggagattcatttgatcaaatcagagcacgcaaaaccgctcccggcatggctttcgttgtggaaagaaggaacggagtcttcatatgatgaatatcaaggcgctggaagacatgatgaaatgcttgcggacggctttgttaaaagcaacgcagatctcatcagacagattttgggcgaacgtgccgctgtaatcggaggtgcatgatgcatgaacgagatcattaagcagtttggagcgccggcaagcggtatgcagttgatctgtttcccgtttgccgggggatattcggcatcattcagaccgcttcacgaacatttaaaaacagattttgatatgctggcgatcgaaccgcctggacacggcacaaatcgaatggcgcttgtcgacaatttggaaaagctcgccgatttgtatatagcggcgcttaagccgcggctttcctctcaattcgtgctgttcggccacagtatgggcggcatggttgtttacaggctggcgcaaaagcttgaacaagaagggatctttccggcagccgctgtgatatcagcgatacagccgccccatattcagcggcaaaaggtgtctcaccaaaaggatgacgcatttctcgaccatctcattcagcttggcggaatacccaaagaactggcagagcacagggaaatgatgaactttttcctgccttcgttcagagcggacta

SEQ ID NO: 18 - protein, 1282 aa, Bacillus licheniformis
LSQFKKEHVQDIYYLSPMQEGMLFHTLLHPGQGFYIEQISMQVKGSFQKDVLEKSMNVIIGRYDIFRTVFVHEKMKRPVQVVLKERSFQAEEIDLSGLSEAEQNERIEDYKRKDKEKGFNLSKDIPMRTAVFKKGQDRYEWVWSYHHILLDGWCFGIVVQELFEVYNALRENRPYSLGPVRPYKEYIKWLESQDKQKSLVYWEHHLAGFEGQTTFSEQRKNPEQAGNQPEELLFTLPKEDTEAFTQLAKAHHTTLSTALQAVWSVLLSRYQRSRDLIFGTVVSGRPADIQGVEHMVGLFINVVPKRATFGPQTTFTELVADLQKQALEAEPHQYVPLYEIQSHIAAPNLIDHIIVFENYPLQEANKQQKEKNLGFTMDEISVFEKSNYDLNLLASPGEEMMLKLAYNRNIFEPSFILRLKEQLLTAIREVIRDPEQPLDDICVVSKKEKERLLHEFNGQVQRKQIGLTIPQWFEQYAEALPEKPAVSAAGRTLTYRELNEKANKVAHLLRKKNVGRGEPVALLFRRSPEMVIAILAVLKAGGAYLPIDPEYPEARIQYMLEDSGAVCMLTQEELAGRAASLSFHKHTILIDDPAVSAESGRNLEIAAEPDDLAYIMYTSGTTGKPKGNLTTHANITRVVKETNYISLSEKDTLLSLSNYAFDGFTFDLYGALLNGAKLVVADQATILHIGKLTEAIQKENITVMFVTTALFNLLVDAGTGWMKGIRKVLFGGERSSVSHVKKAFAAMGPDRIIHVYGPTETTVFATFYPVNRMEDSAVSIPIGKPINETNAYILTENNRLQPIGAVGELCLSGTGVSRGYLNRPELTAEKFAPHPFNSGETMYRTGDLARWLPDGNIDFIGRIDDQVKIRGHRIELGEIEEQLMRCQGVKEAVVTARKSGNGDAALTAYVVPVQGTEVANEEVRRQLARRLPAYMVPAAYIMLEELPLTANGKVNRRLLPEADGRPNSTERRAPRNATEEKLAVIWSEVLGRQQIGIDENFFEIGGHSLKAMAVASRMLKDLAIDVPVNVLFEMPTIEGLAAYIDQGGHVDEVKKTVFNQASTRHLFAFPPVLGYGIMYGKLAEQMPEYRIHAFDFIESDDRAVRYAEAISDLQPEGPLTLIGYSAGCGLAFEVAQALEKNGRKVEKLLMIDSYMKNDVSDLEGRSIEADTAALMEANKDNEYMKIKTVADGIAKKMTAYYSYFVNLVHQGTVSSEIHLIKSEHAKPLPAWLSLWKEGTESSYDEYQGAGRHDEMLADGFVKSNADLIRQILGERAAVIGGA

SEQ ID NO: 19 - DNA, 1678 bp, Bacillus licheniformis
  CDS (501)..(1178)
aacgactttccgatgattcctgtttgttcacggctcggatttgttcggtagtgccggtcgatatgaggcttgagccgattggccaaaaaagcggcatattcgttgcccttcccgccgtactcccgtttattcggcagcaaatgccgcttgggaaaaggcgtgtattcgctttccctgtcttttggagccactccgaccagcagcaattccggcacttcctgactcgaaaacagcttttcgattgagcgcagactgttgctgtatgccggatcgaataaatctcccaggtcatggacgtatagaacgggatagcggttttcaccgctcccgtatgaaggaggaagatagatcaaaagctccctcccgaagcatgtatcttttatcaatgttccgtctatcatgttcattactccattctgcacaagtatcatgcctatctttcctgctgtctgtttgctaaaatgaaagcagaaaaatatcatgtgaagaggttgcgtttgatgaagatttacgccgtttatatggatcagctgccggaagccggtcaatttaaccgaatgatggccatggtgtctgaagaaaagcgaaaaaaagtgggcagatacagatatcccgaagatgccagccggacgctgatcggagaagtgctggcgaggcacatcatcagcgaaacgtttcatttgccgaatgagcagatccgttttacagaaggaaggtacggcaaaccggctgctgaaggtctgaacgactttcatttcaacatttcccactccggaaaatggatcgtgtgcggagccggcgcgcagccgatcggagtggatgtcgaaaagatcaagccgatcaatttcgacattgccaaacggtttttttcaccttccgagcacgatgatctcatggaaaaggacgaatcagaacgcctgtcgtatttctatcatttgtggacgatgaaagaaagctttattaaacaggcgggaaaaggcctgtctctgccgcttgattccttctccgtcaaactgaacgaacaggggcgagcttcggtggaaactccccccggctatccgccttgctatatacaagcctatgaaattgaccccggctataaaatggcggtgtgttccgccgcacctgaatttcccggccggattgtgatgaaaagctgcgctgaactgtcaatgctttaaaaagtcccgccctaaaaataagcggtttccaccgcttgccgcaaccgctcgatgccgctgcggatgtcctcttccttcacattggacgtattgattttcaatagatttctttttggaaaagccggcatataatgagcgttgaccggttcgagcaggacgtcccgtttttgtaggcttttaatcagacgggatgccgggatcgtccggtccaattcaatatgggtgtggatagccggctgtgcgggaggatgaaattcaaacacatcccctagagacgcggaacattcttttagcagatcatgcagaaggcatgatcttttttgataggaagaacggatcttttgcttatgcttttcaaacatcccgctcttcaaatagatctccaaagcggcctgagacagcatggaactgtcaatgtcgagcagctgtttgtaccggttgaacacaattgtcagagaatcgggcagcacggcggctccgacccgcagacccggaaa

SEQ ID NO: 20 - protein, 226 aa, Bacillus licheniformis
MKIYAVYMDQLPEAGQFNRMMAMVSEEKRKKVGRYRYPEDASRTLIGEVLARHIISETFHLPNEQIRFTEGRYGKPAAEGLNDFHFNISHSGKWIVCGAGAQPIGVDVEKIKPINFDIAKRFFSPSEHDDLMEKDESERLSYFYHLWTMKESFIKQAGKGLSLPLDSFSVKLNEQGRASVETPPGYPPCYIQAYEIDPGYKMAVCSAAPEFPGRIVMKSCAELSML

SEQ ID NO: 21 - DNA, 1738 bp, Bacillus licheniformis
  CDS (501)..(1238)
cgctgacattgatcggctattcagccggctgcggacttgcctttgaagtggcgcaagcgctcgaaaagaacggacggaaggtcgaaaagctgctgatgatcgactcatatatgaagaacgatgtcagcgatttagaagggcggtcaattgaagccgatacggccgccttgatggaagccaacaaggacaatgaatatatgaaaatcaagacggttgccgatggcatcgctaaaaagatgacagcctattattcttactttgtcaatcttgtccatcaaggaacggtcagctcggagattcatttgatcaaatcagagcacgcaaaaccgctcccggcatggctttcgttgtggaaagaaggaacggagtcttcatatgatgaatatcaaggcgctggaagacatgatgaaatgcttgcggacggctttgttaaaagcaacgcagatctcatcagacagattttgggcgaacgtgccgctgtaatcggaggtgcatgatgcatgaacgagatcattaagcagtttggagcgccggcaagcggtatgcagttgatctgtttcccgtttgccgggggatattcggcatcattcagaccgcttcacgaacatttaaaaacagattttgatatgctggcgatcgaaccgcctggacacggcacaaatcgaatggcgcttgtcgacaatttggaaaagctcgccgatttgtatatagcggcgcttaagccgcggctttcctctcaattcgtgctgttcggccacagtatgggcggcatggttgtttacaggctggcgcaaaagcttgaacaagaagggatctttccggcagccgctgtgatatcagcgatacagccgccccatattcagcggcaaaaggtgtctcaccaaaaggatgacgcatttctcgaccatctcattcagcttggcggaatacccaaagaactggcagagcacagggaaatgatgaactttttcctgccttcgttcagagcggactacagagcgcttgagacgttctgccataccgatcatacgccgcttagatcgcctgtgcatattctgaacggagaccgcgatgagaaatgcatgagggatgcgcttggctggaaaaagtgggcgaatacgatcgatttccactatttcaaaggcggacatatgtatcttttatcagaaacggaacaagtagccgccaagttgaaaacgattgccgcggccagccgcagtacggctgttccgacaatgtaaagaaagcggaaaatcggtgtgctgcagctgccgattttccgttttttcagctttttttcagccgattttcccgccgaaccatcgtcgccccaataattccggcaatgatcatccccgttccggcgaagtgaaaccattcgagtgattctcccagccatataatccccgccaaaatcgtcaccagtgtcgccaagtgattaaacatgctgattttcgcagcttctatttttgacagtgcataatttgataaaaatgatgtcgcaaacgatgacaacacgcctaaatacaataccgaaagaacaaacaccggatcagtgaagggggcaaaaaatccgctcagcgtgccgtctgacatatggctgattccggcgatgccgttgaaaaaaagaaatcccgctcccgttgtgacatatgttaattcaagcgagctgaaggttcgcgtcatctttctggcgagcacgctgtagcccgccgatgaaagcgccgaaagcaaaatc

SEQ ID NO: 22 - protein, 246 aa, Bacillus licheniformis
MNEIIKQFGAPASGMQLICFPFAGGYSASFRPLHEHLKTDFDMLAIEPPGHGTNRMALVDNLEKLADLYIAALKPRLSSQFVLFGHSMGGMVVYRLAQKLEQEGIFPAAAVISAIQPPHIQRQKVSHQKDDAFLDHLIQLGGIPKELAEHREMMNFFLPSFRADYRALETFCHTDHTPLRSPVHILNGDRDEKCMRDALGWKKWANTIDFHYFKGGHMYLLSETEQVAAKLKTIAAASRSTAVPTM